(19) United States
(12) Patent Application Publication
(10) Pub. No.: US 2023/0158325 A1
(43) Pub. Date: May 25, 2023

(54) METHODS AND APPARATUS FOR MEDICAL TREATMENT OF PATIENT TISSUES

(71) Applicant: Patrick Gooi, Calgary (CA)

(72) Inventor: Patrick Gooi, Calgary (CA)

(21) Appl. No.: 17/992,729

(22) Filed: Nov. 22, 2022

Related U.S. Application Data
(60) Provisional application No. 63/282,004, filed on Nov. 22, 2021.
(60) Provisional application No. 63/320,800, filed on Mar. 17, 2022.

(51) Int. Cl. A61N 5/06; A61F 9/00; A61K 31/525

(52) U.S. Cl. CPC A61N 5/062; A61F 9/0017; A61K 31/525; A61N 2005/0661

(57) ABSTRACT

A collagen cross-linking system for treating a tissue of a patient, the system including: a catheter having a flexible shaft and a conforming member, the flexible shaft having distal and proximal ends, a shaft body extending between the distal end and the proximal end, the shaft body defining a lumen, the conforming member being fixed to the catheter shaft near the distal end, the conforming member comprising a membrane defining a cavity in fluid communication with the lumen; a fluid source coupled to the proximal end of the catheter, the flexible shaft of the catheter being configured so that a flow of photosensitizing fluid provided by the fluid source flows through the membrane of the conforming member; and a light source coupled to the proximal end of the catheter, the catheter being configured so that photo-activating light generated by the light source passes through the membrane of the conforming member.

### RELATED APPLICATIONS

This patent application claims the benefit of U.S. Provisional Application No. 63/282,004, filed Nov. 22, 2021, and U.S. Provisional Application No. 63/320,800, filed Mar. 17, 2022, the disclosures of which are incorporated by reference in their entireties.

### FIELD OF THE INVENTION

The present invention is directed to systems, methods and devices for medically treating patient tissues, and in particular, tissues of the human eye.

### BACKGROUND

A number of surgical procedures for treating the tissue of an eye are known, including surgical procedures for treating glaucoma. For example, glaucoma filtering surgery or “trabeculectomy” is a known procedure for treating glaucoma. However, known surgical procedures may not always yield a bleb or capsule that is of the optimal size, configuration, and density surrounding tissue due to variability in surgical techniques and patient healing factors.

### SUMMARY

Embodiments of the present disclosure address shortcomings of known surgical procedures for treating glaucoma.

In an embodiment, the present invention is a collagen cross-linking system for treating a tissue of a patient. The system includes a catheter, a fluid source and a light source. The catheter comprises a flexible shaft and a conforming member, the flexible shaft having a distal end, a proximal end, a shaft body extending between the distal end and the proximal end, the shaft body defining a lumen, the conforming member being fixed to the catheter shaft near the distal end, the conforming member comprising a membrane defining a cavity in fluid communication with the lumen. The fluid source is operatively coupled to the proximal end of the catheter, the flexible shaft of the catheter being adapted and configured so that a flow of photosensitizing fluid provided by the fluid source flows through the membrane of the conforming member. The light source is operatively coupled to the proximal end of the catheter, the catheter being adapted and configured so that photo-activating light generated by the light source passes through the membrane of the conforming member.

In another embodiment, the present invention is directed to a method for the medical treatment for treating a target tissue in a patient's body. The method comprises the following steps:
positioning a conforming member near the target tissue, the conforming member comprising a membrane;
directing a flow of photosensitizing fluid to impinge upon the target tissue, wherein the photosensitizing fluid flows through the membrane of the conforming member; and
irradiating the target tissue with photo-activating light that passes through the membrane of the conforming member.

In another embodiment, the present invention is directed to an implantable aqueous humor wicking device for transmission of aqueous humor in an eye of a patient. The device comprises a tube having an anterior end configured to interface with an anterior chamber of the eye of the patient, and a posterior end, the tube defining a flow channel for transmission of the aqueous humor of the eye of the patient from the anterior chamber to the posterior end of the tube. The device also comprises a wicking portion coupled to the posterior end of the tube and configured to absorb and retain aqueous humor received from the tube, the wicking portion comprising a biocompatible absorbent woven material, and defining an interior cavity receiving the posterior end of the tube. The biocompatible absorbent woven material of the wicking portion is configured to cause the aqueous humor to flow from the anterior chamber to the wicking portion due to a capillary action produced by the absorbent woven material.

### BRIEF DESCRIPTION OF THE DRAWINGS

The drawings included in the present patent application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 is a stylized representation of a medical procedure in accordance with the detailed description.

FIG. 2 is a stylized perspective view showing the eye of a patient.

FIG. 3 is a stylized cross-sectional view taken along section plane shown in FIG. 2.

FIG. 4 is a stylized diagram view showing a human face including a pair of eyes.

FIG. 5A is a stylized diagram showing four regions an eye.

FIG. 5B is a stylized diagram showing a region of an eye.

FIG. 6A is a stylized diagram showing an example incision in an eye.

FIG. 6B is a stylized diagram showing a collagen cross-linked region of an eye.

FIG. 7A is a stylized diagram showing an example peritomy incision in an eye.

FIG. 7B is a stylized diagram showing a trabeculectomy pocket in an eye.

FIG. 8 is a stylized diagram showing an example single quadrant peritomy in an eye.

FIG. 9 is a stylized diagram showing an example treatment pattern for use with methods and apparatus in accordance with this detailed description.

FIG. 10 is a stylized diagram showing an additional example treatment pattern for use with methods and apparatus in accordance with this detailed description.

FIG. 11 is a stylized plan view illustrating an example collagen crosslinking system in accordance with this detailed description.

FIG. 12 illustrates an example collagen crosslinking system in accordance with this detailed description.

FIG. 13A is a stylized cross-sectional view showing an eye.

FIG. 13B is an enlarged, stylized cross-sectional view showing a portion of the eye illustrated in FIG. 13A.

FIG. 13C is an enlarged, stylized cross-sectional view showing a portion of FIG. 13B.

FIG. 14 is a stylized diagram illustrating an example method of treatment performed on an eye, with the eye shown as a cross-sectional view.

FIG. 15 is a stylized diagram illustrating an example method of treatment performed on an eye.

FIG. 16 is a stylized diagram showing an eye. An incision has been made in the tissue of the eye in the embodiment of FIG. 16.

FIG. 17A and FIG. 17B are stylized diagrams illustrating a medical procedure in accordance with this detailed description. In FIG. 17A, an incision has divided a tissue into a first portion and a second portion. In FIG. 17B, collagen crosslinking across the incision is illustrated using a pattern of cross-hatched lines.

FIG. 18A and FIG. 18B are stylized diagrams illustrating a medical procedure in accordance with this detailed description. The stylized diagram of FIG. 18A includes a cross-sectional view of a region of tissue defining a fluid flow channel. In FIG. 18B collagen crosslinking in the tissue is illustrated using a pattern of cross-hatched lines.

FIG. 19 is a stylized diagram illustrating an aqueous humor wicking device in a perspective view, according to an embodiment.

FIG. 20 is a view in cross section of the aqueous humor wicking device of FIG. 19.

FIG. 21 is a stylized diagram illustrating a barbed portion of the aqueous humor wicking device of FIGS. 19-20.

FIG. 22 is a stylized diagram illustrating an embodiment of an aqueous humor wicking device with an aqueous flow direction indicator.

FIG. 23 is a stylized diagram illustrating placement of a portion of an aqueous humor wicking device between tissue layers of an eye.

FIG. 24 is a stylized diagram of an embodiment of a wicking device.

FIG. 25 is a stylized diagram illustrating fluid flow out of an aqueous humor wicking device.

FIG. 26 is a stylized diagram illustrating embodiment of a tubeless aqueous humor wicking device in fluid communication with the eye.

FIG. 27 is a stylized diagram of a portion of the device of FIG. 25 in fluid communication with the anterior chamber of the eye.

FIG. 28 is a stylized diagram illustrating another embodiment of an aqueous humor wicking device.

FIG. 29 is a stylized diagram illustrating a cytokine-adsorbent wicking device in cross section as implanted into an eye, according to an embodiment.

FIG. 30 is a stylized diagram illustrating a cytokine-adsorbent wicking device in cross section, according to another embodiment.

FIG. 31 is a front perspective view of a cytokine-adsorbent device, according to an embodiment.

FIG. 32 is a stylized diagram illustrating an aqueous filtering system in cross section as implanted into an eye, according to an embodiment.

While the embodiments of the disclosure are amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

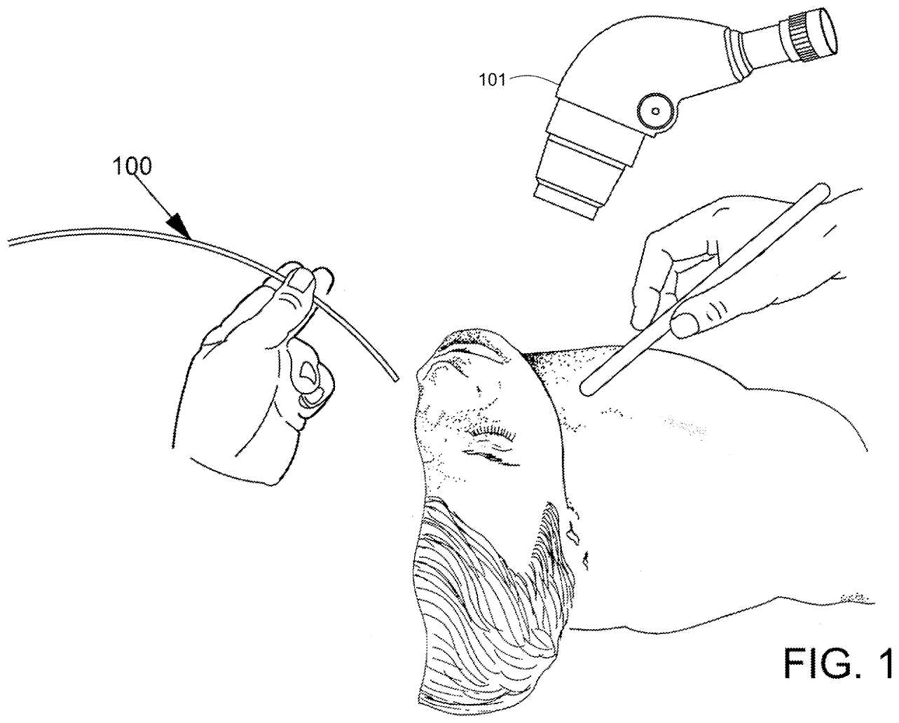

FIG. 1 is a stylized representation of a medical procedure in accordance with this detailed description. In the example procedure of FIG. 1, a physician is treating the eye of a patient. The left hand of the physician is shown holding a portion of a catheter 100 in the example embodiment of FIG. 1. In some example methods, a treatment procedure may include making incisions using a scalpel. The catheter 100 may be used to promote collagen crosslinking across incisions in tissue to promote healing of the tissue in some example methods. In some example methods, a conformer may be used to arrange the tissue in a desired configuration. In some example methods, a conformer may be used to apply pressure to tissue during collagen crosslinking. In some example embodiments, the conformer may comprise an inflatable member. During the procedure illustrated in FIG. 1, the physician may view the eye of the patient using a microscope 101.

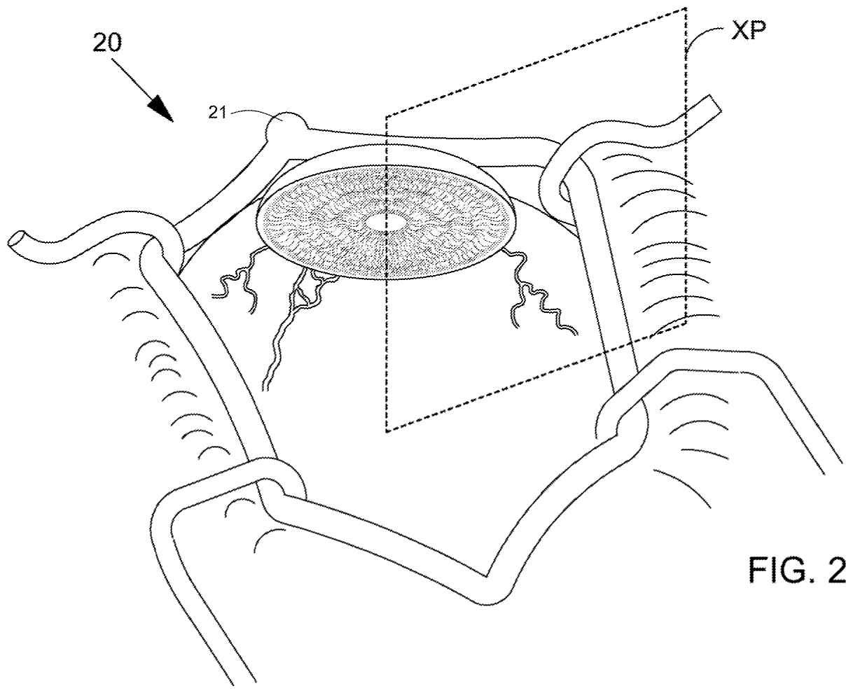

FIG. 2 is a stylized perspective view showing the eye 20 of a patient. In the embodiment of FIG. 2, the upper and lower eyelids of the eye are held open with surgical tools 21 so that the eye is accessible to the physician. The cornea of the eye 20 meets the sclera of the eye 20 at the limbus. The conjunctiva of the eye 20 is loose connective tissue that covers the surface of the eyeball (bulbar conjunctiva) and doubles back upon itself to form the inner layer of the eyelid (palpebral conjunctiva). The conjunctiva is firmly adhered to the sclera at the limbus, where it meets the cornea.

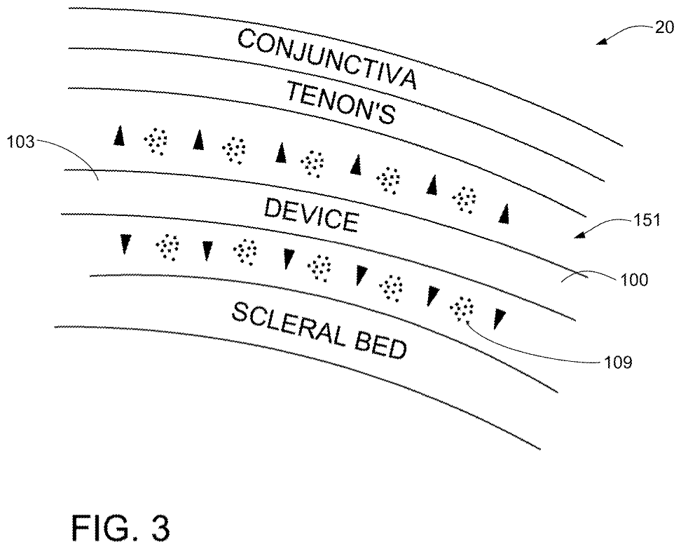

FIG. 3 is a stylized cross-sectional view taken along the plane XP shown in FIG. 2. With reference to FIG. 2, it will be appreciated that the plane XP (shown with dashed lines) intersects the sclera of the eye 20. With reference to FIG. 3, it will be appreciated that a portion of a device is located between the conjunctiva and the scleral bed of the eye. In an embodiment, the device may be a distal end 103 of a catheter 100, as described further below. The Tenon capsule is also visible in FIG. 3. The Tenon capsule is a thin membrane that envelops the eyeball from the optic nerve to the corneal limbus and forming a socket in which the eye moves.

An example therapy method illustrated in FIG. 3 includes delivering a flow of photosensitizing fluid 109 to a target region of tissue 151 and irradiating the target region with photo-activating light. In the stylized diagram of FIG. 3, photo-activating light emitted from the device is illustrated using solid triangles. The flow of photosensitizing fluid exiting the device is illustrated using solid circles 109 in the stylized diagram of FIG. 3. In some example embodiments, the photosensitizing fluid 109 comprises riboflavin. In some example embodiments, the photosensitizing fluid comprises oxygen.

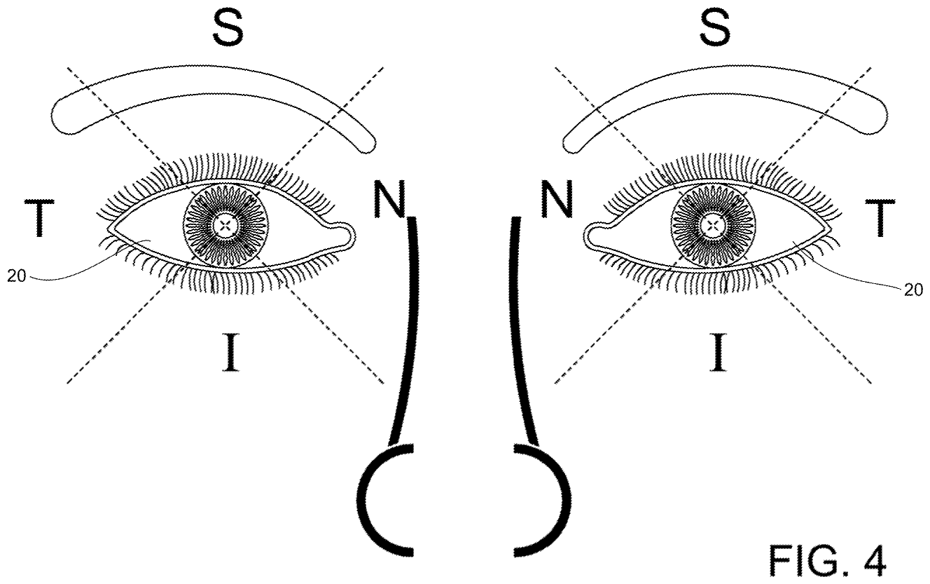

FIG. 4 is a stylized diagram view showing a human face including a pair of eyes 20. For purposes of illustration, each eye 20 is divided into four portions using dashed lines in FIG. 4. The four portions are labeled S, I, N, and T. In the example embodiment of FIG. 4, portion S is a superior portion of each eye and portion I is an inferior portion of each eye. Portion N is a nasal portion of each eye and portion T is a temporal portion of each eye in the example embodiment of FIG. 4.

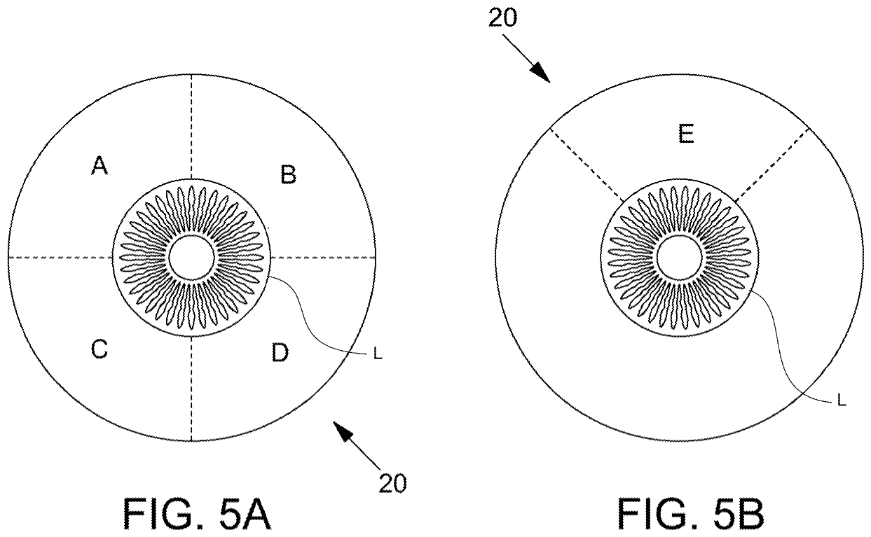

FIG. 5A is a stylized diagram showing four regions an eye 20. The four regions are labeled A, B, C, and D in FIG. 5A. In the example embodiment of FIG. 5A, region A corresponds to a superotemporal quadrant of the eye and region C corresponds to an inferotemporal quadrant of the eye. Region B corresponds to a superonasal quadrant of the eye and region D corresponds to an inferonasal quadrant of the eye in the example embodiment of FIG. 5A.

FIG. 5B is a stylized diagram showing a region E of an eye 20. In the example embodiment of FIG. 5B, with an apex of the region E is located toward the limbus of the eye. In the example embodiment of FIG. 5B, region E has a shape that fans out as region E extends away from the limbus L of the eye.

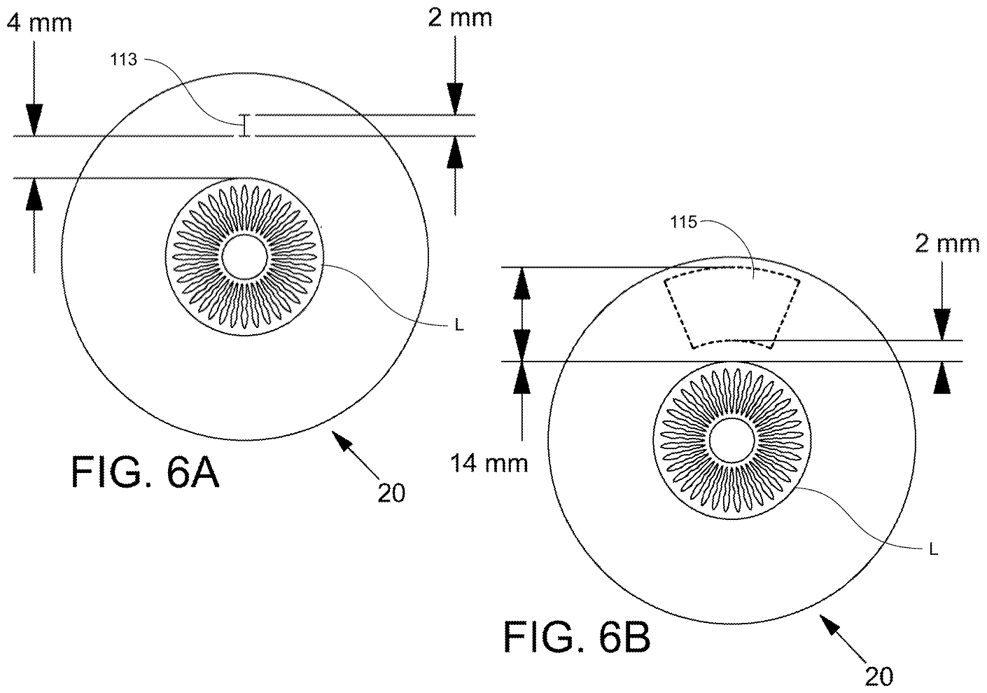

FIG. 6A is a stylized diagram showing an example incision 113 in an eye 20. In the example embodiment of FIG. 6A, the incision 113 begins at a location 4 mm from the limbus L of the eye 20. The incision has a length of 2 mm in the example embodiment of FIG. 6A. FIG. 6B is a stylized diagram showing a collagen cross-linked region of an eye 20. Example boundaries of the collagen cross-linked region 115 are illustrated using dashed lines in FIG. 6B. In the example embodiment of FIG. 6B, the collagen cross-linked region has an inner boundary located 2 mm from the limbus L of the eye 20. The collagen cross-linked region 115 has an outer boundary located 14 mm from the limbus L of the eye 20 in the example embodiment of FIG. 6B.

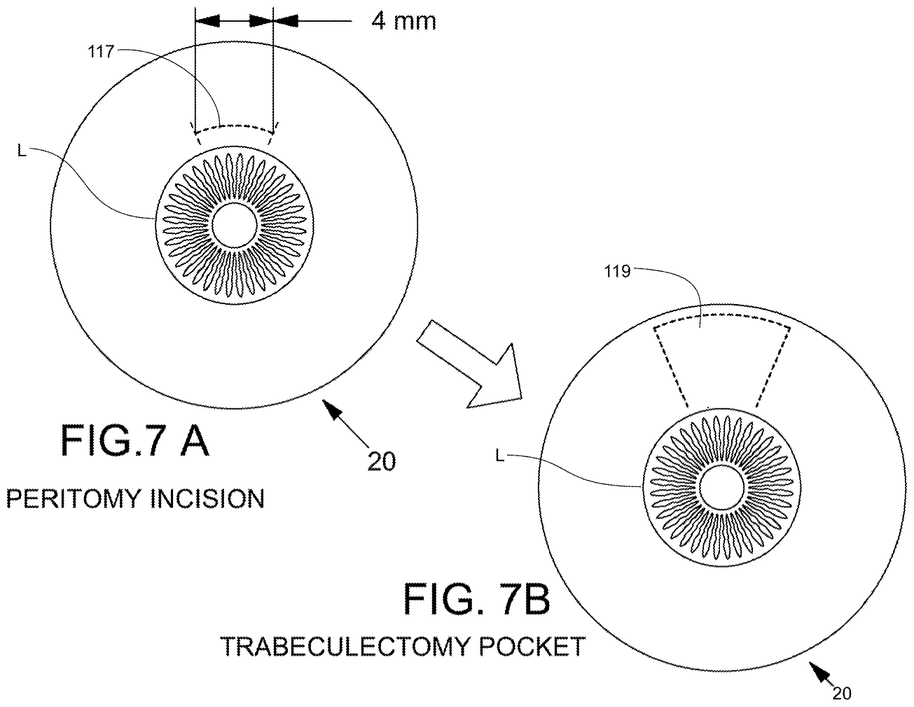

FIG. 7A is a stylized diagram showing an example peritomy incision in an eye 20. In the example embodiment of FIG. 7A, the peritomy incision 117 has an arcuate shape that is offset from the limbus L of the eye 20. The peritomy incision 117 has a length of 4 mm in the example embodiment of FIG. 7A. FIG. 7B is a stylized diagram showing a trabeculectomy pocket 119 in an eye 20. Example boundaries of the trabeculectomy pocket are illustrated using dashed lines in FIG. 7B.

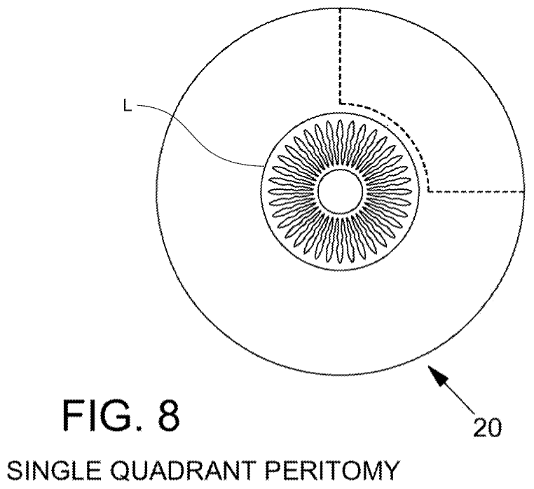

FIG. 8 is a stylized diagram showing an example single quadrant peritomy in an eye 20. In the example embodiment of FIG. 8, the single quadrant peritomy includes an arcuate portion that is off set from the limbus of the eye. Example methods in accordance with this detailed description may include implanting a device in the eye of a patient. Examples of devices that may be suitable in some applications include tube shunts (e.g., the PRESERFLO MicroShunt marketed by Santen Pharmaceutical Co. Ltd.), glaucoma implants (e.g., the BAERVELDT Glaucoma Implant marketed by Johnson and Johnson) and other devices used to treat glaucoma (e.g. the Ahmed valve).

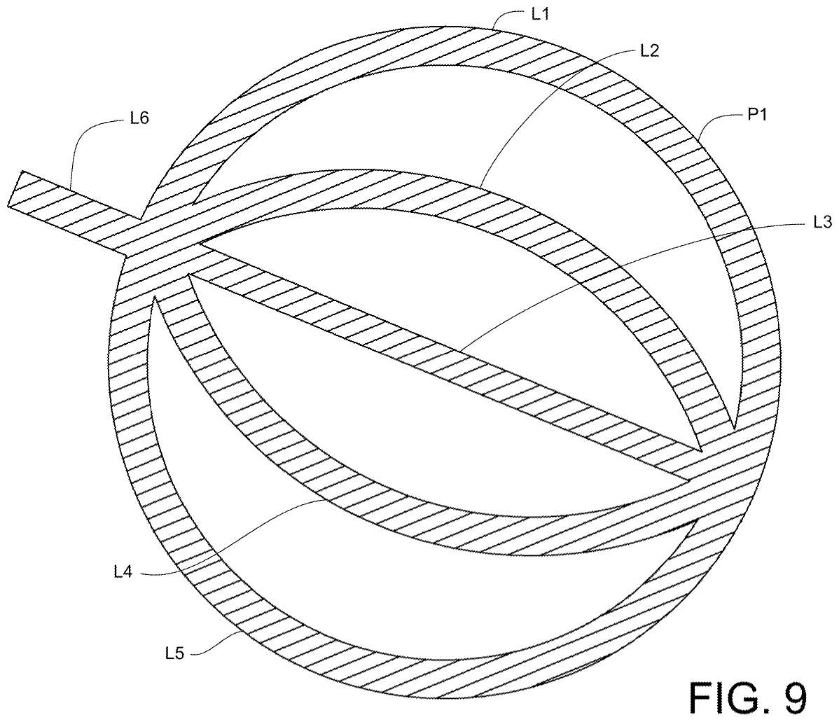

FIG. 9 is a stylized diagram showing an example treatment pattern P1 for use with methods and apparatus in accordance with this detailed description. In example embodiment of FIG. 9, the example treatment pattern P1 has a shape that is analogous to the pattern of lines seen on a pumpkin. The treated areas of tissue are illustrated by a pattern of crosshatch lines L1 to L5 and line L6 in FIG. 9. With reference to FIG. 9, it will be appreciated that the treatment pattern includes areas of untreated tissue that are encircled by areas of treated tissue.

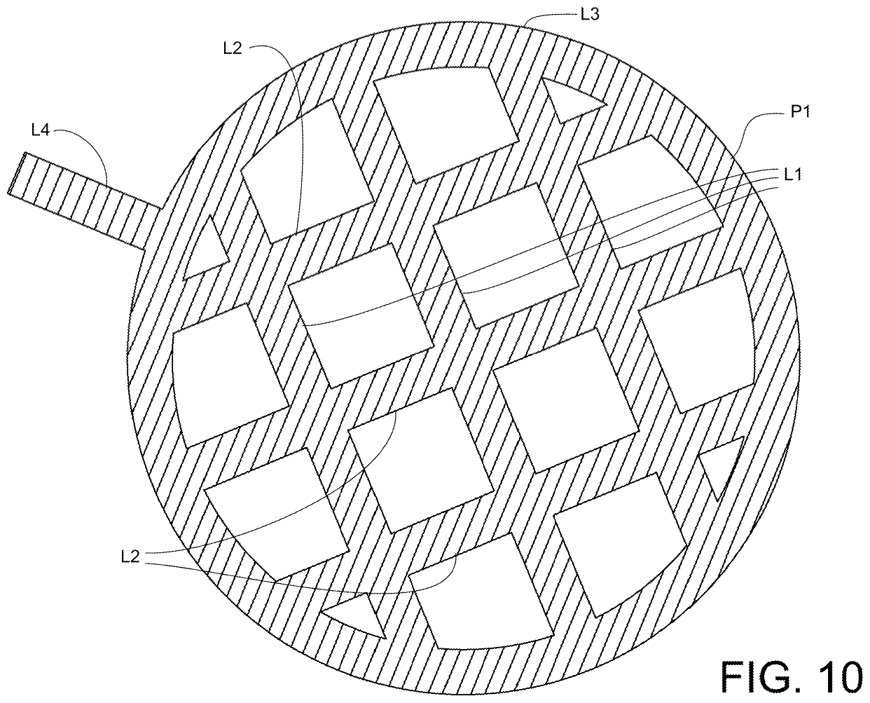

FIG. 10 is a stylized diagram showing an additional example treatment pattern P2 for use with methods and apparatus in accordance with this detailed description. The treated areas of tissue are illustrated by a pattern of crosshatch lines L1 and L2, circumferential line L3 and line L4, in FIG. 10. With reference to FIG. 10, it will be appreciated that the treatment pattern includes untreated areas of tissue that are encircled by treated areas of tissue. In example embodiment of FIG. 10, the example treatment pattern includes a plurality of lines that cross each other in a crisscross pattern.

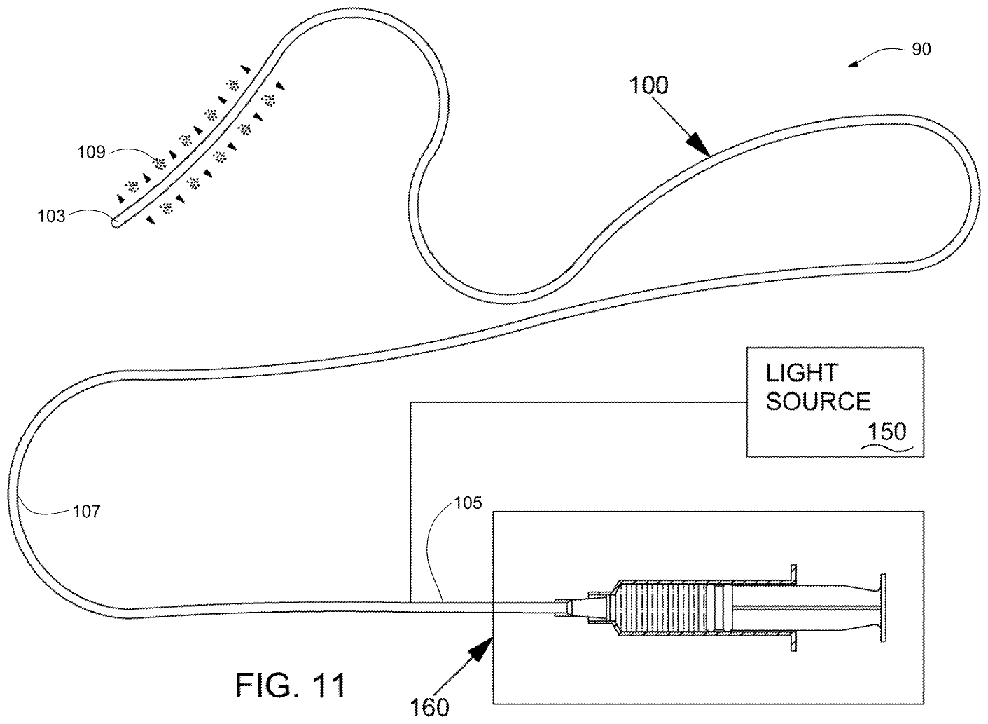

FIG. 11 is a stylized plan view illustrating an example collagen crosslinking system 90 in accordance with this detailed description. The system of FIG. 11 includes a catheter 100, a light source 150 and a fluid flow source 160. In the example embodiment of FIG. 11, the catheter has a distal end 103, a proximal end 105, and a flexible shaft 107 extending between the distal end and the proximal end. The fluid flow source 160 is operatively coupled to the shaft of the catheter in the example embodiment of FIG. 11.

In some example embodiments, the flexible shaft 107 of the catheter 100 is adapted and configured so that a flow of photosensitizing fluid 109 provided by the fluid flow source 160 is delivered to a location proximate the distal end 103 of the catheter 100. The flow of photosensitizing fluid 109 exiting a distal portion 103 of the catheter 100 is illustrated using solid circles in the stylized diagram of FIG. 11. In an embodiment, the photosensitizing fluid 109 may be dispensed through apertures in, or a membrane of, flexible shaft 107 at distal end 103. In some example embodiments, the photosensitizing fluid 109 comprises riboflavin. In some example embodiments, the photosensitizing fluid 109 comprises oxygen.

In the example embodiment of FIG. 11, the light source 150 is operatively coupled to the shaft 107 of the catheter 100. In some embodiments, the flexible shaft 107 of the catheter 100 is adapted and configured so that photo-activating light generated by the light source 150 is emitted from a distal portion 103 of the catheter 100. In the stylized diagram of FIG. 11, photo-activating light emitted from the catheter 100 is illustrated using solid triangles. In some example embodiments, the photo-activating light provided by the light sources 150 comprises ultraviolet light. In some example embodiments, the light source 150 generates light having a wavelength of 370 nm. In some embodiments, the light exits through apertures in, or light-permeable membrane of, distal end 103 of flexible shaft 107.

In some embodiments, the catheter shaft 107 is dimensioned and configured such that the distal end 103 of the catheter 100 can enter the body of a patient through a small incision, such as one of the incisions described above, and delivery therapy to a location inside the body. In some embodiments, a system 90 in accordance with this detailed description provides the ability to precisely target tissue therapy that promotes collagen crosslinking in the target area of a tissue.

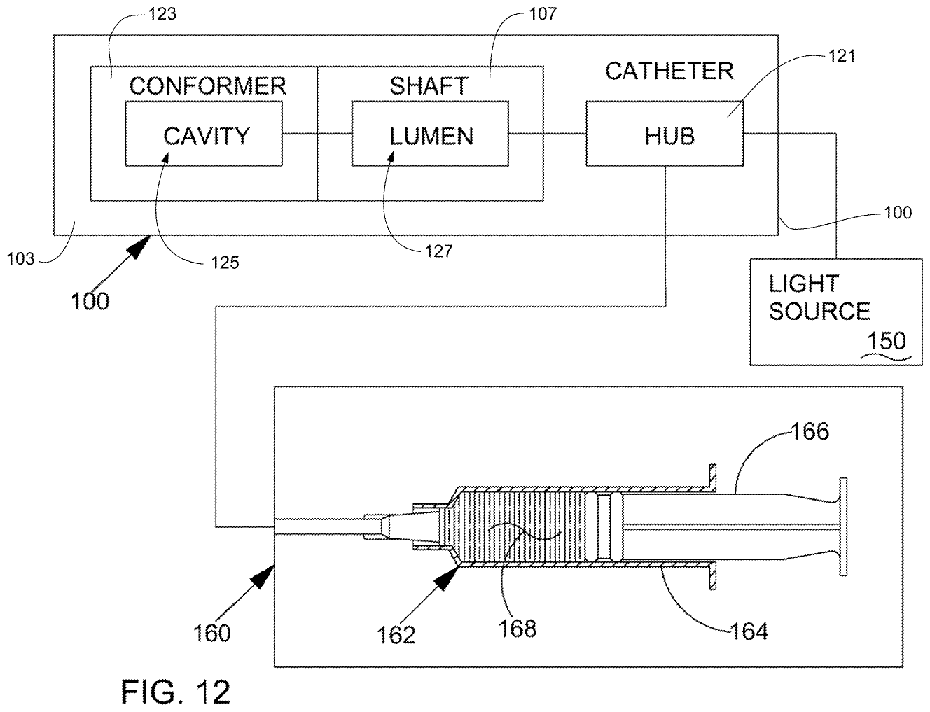

FIG. 12 illustrates an example collagen crosslinking system in accordance with this detailed description. The illustration shown in FIG. 12 is not meant to imply physical or architectural limitations to the manner in which an illustrative embodiment may be implemented. Other components in addition to or in place of the ones illustrated may be used. Some components may be optional. Also, some blocks may illustrate functional components. One or more of these blocks may be combined, divided, or combined and divided into different blocks in some implementations. The example system shown in FIG. 12 includes a fluid flow source 160 capable of providing a flow of fluid. The fluid flow source 160 comprises a syringe 162 in the example embodiment of FIG. 12. In the example embodiment of FIG. 12, the syringe 162 comprises a syringe barrel 164 and a syringe plunger 166, a distal portion of the syringe plunger 166 is slidingly received in the syringe barrel 164. The syringe barrel 164 and the syringe plunger 166 cooperate to define the fluid chamber 168 in the embodiment of FIG. 12. In some example embodiments, the fluid flow source 160 may include an actuator that selectively causes the syringe 162 to expel fluid from the fluid chamber 168. Fluid flow sources that may be suitable in some applications are disclosed in the following United States patents all of which are hereby incorporated by reference herein: U.S. Pat. Nos. U.S. Pat. Nos. 9,113,843, 9,220,834, 9,241,641, 9,333,293, 9,352,105, and 9,457,140.

The collagen crosslinking system of FIG. 12 includes a catheter 100 having a distal end 103, a proximal end 105, and a flexible shaft 107 extending between the distal end and the proximal end. The catheter 100 includes a hub 121 that is fixed to the catheter shaft 107 near the proximal end of the catheter. The fluid flow source of the system is operatively coupled to the shaft of the catheter via the hub 121 in the example embodiment of FIG. 12. In the example embodiment of FIG. 12, a light source 150 is also operatively coupled to the shaft 107 of the catheter 100 via the hub 121. In some embodiments, the flexible shaft 107 of the catheter 100 is adapted and configured so that photo-activating light generated by the light source 150 is emitted from a distal portion 103 of the catheter 100.

In the example embodiment of FIG. 12, the catheter 100 includes a conformer 123 that is fixed to the distal end 103 of the catheter shaft 107. The conformer defines a cavity 125 that is disposed in fluid communication with a lumen 127 defined by the catheter shaft 107 in the example embodiment of FIG. 12. In some example embodiments, the conformer 123 comprises an inflatable member. In some example methods, a conformer 123 may be used to arrange the tissue in a desired configuration. In some example methods, a conformer 123 may be used to apply pressure to tissue during collagen crosslinking. In some example embodiments, the conformer 123 comprises a light permeable membrane and photo-activating light generated by the light source passes through the membrane of the conformer. In some example embodiments, the conformer 123 comprises a fluid permeable membrane and a flow of photosensitizing fluid 109 provided by the fluid flow source 160 passes through the membrane of the conformer 123.

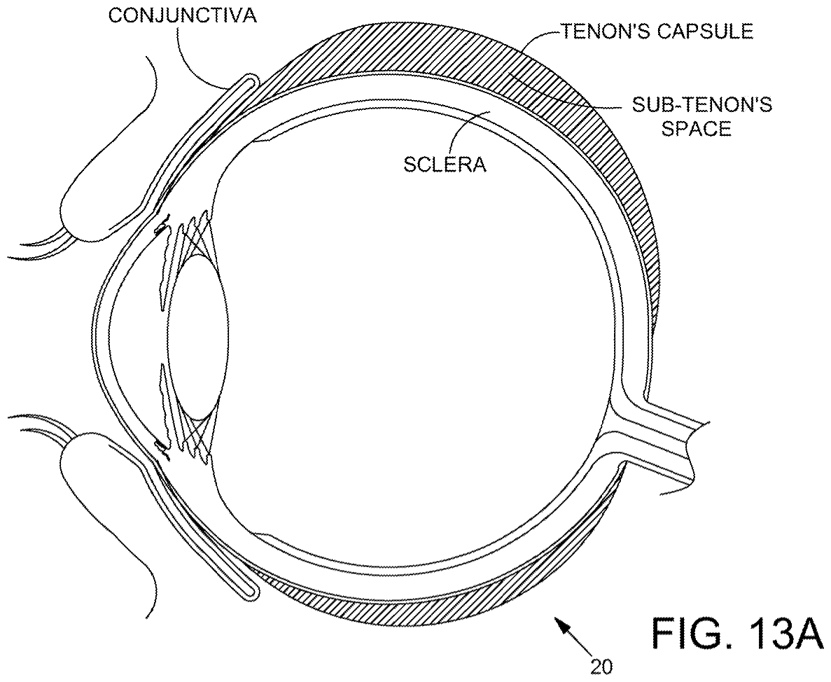

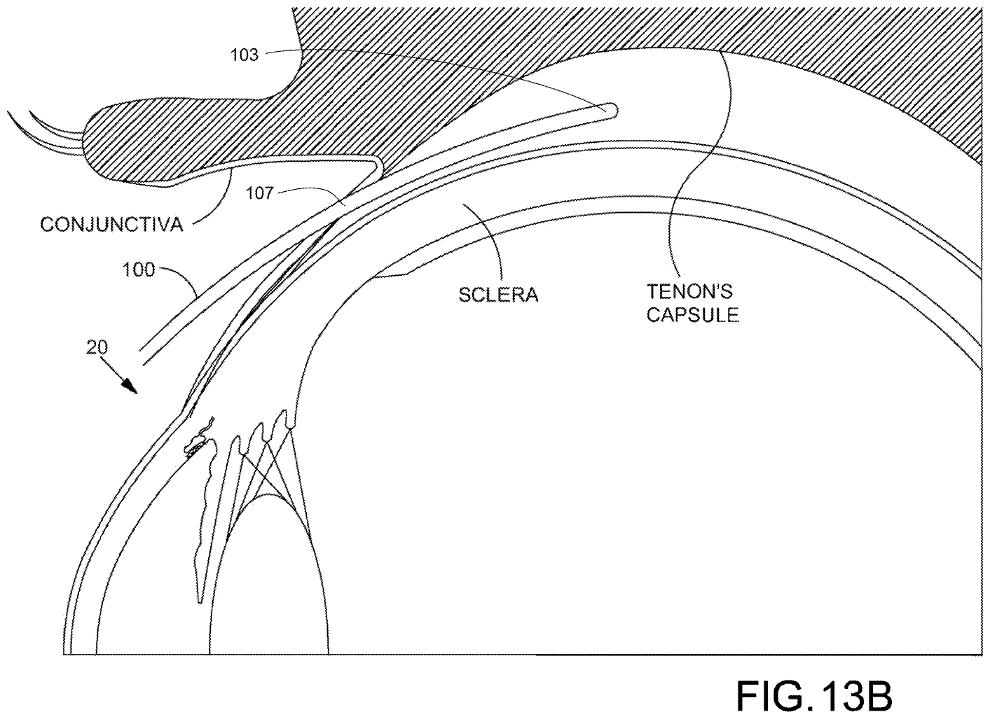

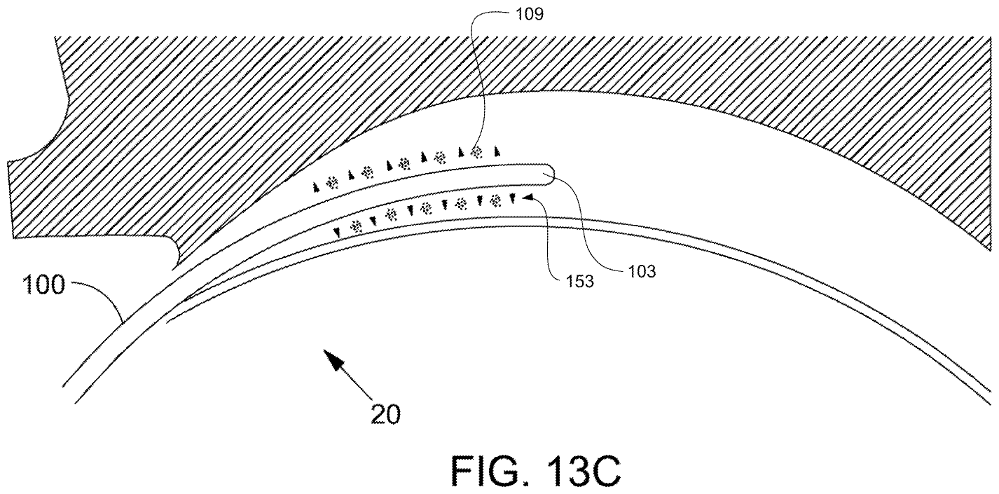

FIG. 13A, FIG. 13B and FIG. 13C are stylized cross-sectional views showing an eye 20. The Tenon's capsule of the eye is visible in FIG. 13. The Tenon's capsule is a thin membrane that envelops the eyeball from the optic nerve to the corneal limbus and forming a socket in which the eye moves. The Sub-tenon's space of the eye is shown using a pattern of crosshatch lines in FIG. 13. With reference to FIG. 13, it will be appreciated that the sub-Tenon's space is located between the Tenon's capsule and the sclera of the eye.

FIG. 13B is an enlarged cross-sectional view showing a portion of the eye 20 illustrated in FIG. 13A. It will be appreciated that the shaft 107 of a catheter 100 is extending between the conjunctiva and the scleral bed of the eye 20. In the embodiment of FIG. 13B, a distal portion 103 of the catheter 100 is located in the sub-Tenon's space of the eye 20. The catheter shaft 107 can be seen extending between the conjunctiva and the scleral bed of the eye 20 in FIG. 13B. The conjunctiva of the eye 20 a loose connective tissue that covers the surface of the eyeball (bulbar conjunctiva) and doubles back upon itself to form the inner layer of the eyelid (palpebral conjunctiva). The conjunctiva is firmly adhered to the sclera at the limbus, where it meets the cornea. The catheter shaft can be seen extending between the Tenon's capsule and the sclera of the eye 20 in FIG. 13B.

FIG. 13C is an enlarged cross-sectional view showing a portion of FIG. 13B. Example therapy methods in accordance with this detailed description may include delivering a flow of photosensitizing fluid 109 to a target region 153 of tissue and irradiating the target region 153 with photo-activating light. In the stylized diagram of FIG. 13, photo-activating light emitted from a distal portion a catheter 100 is illustrated using solid triangles and a flow of photosensitizing fluid exiting the distal portion of the catheter 100 is illustrated using solid circles. With reference to FIG. 13C, it will be appreciated that a distal portion of the catheter 100 is located in Tenon's capsule of the eye 20.

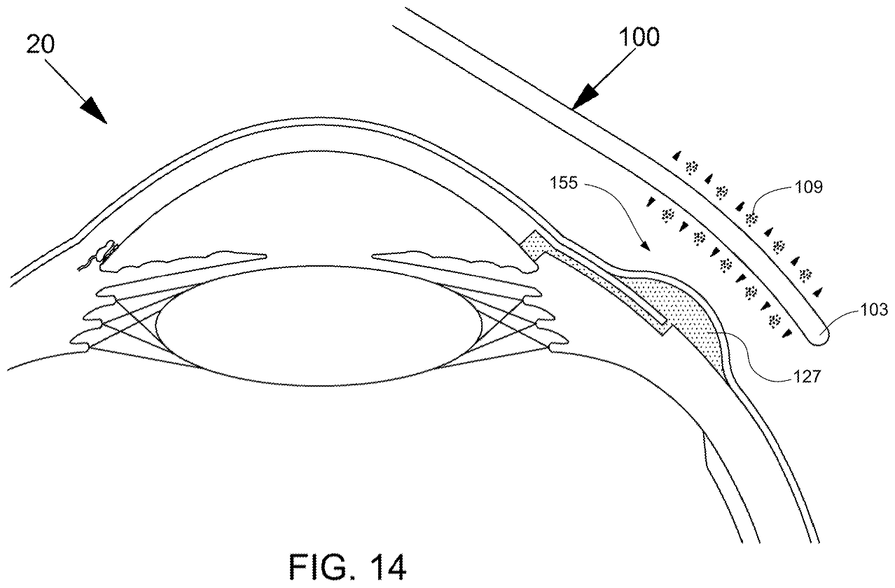

FIG. 14 is a stylized diagram illustrating an example method of treatment performed on an eye 20. The eye 20 is shown as a cross-sectional view in FIG. 14. In the example method illustrated in FIG. 14, eye 20 is the eye of a patient being treated patient for glaucoma. In some example methods, glaucoma may be treated, by forming a pathway that allows aqueous humor to flow out of the anterior chamber of the eye. In some example methods, glaucoma may be treated, by forming a pathway that allows aqueous humor to flow into a cavity defined by a bleb. An example bleb 127 is shown in the cross-sectional view of FIG. 14. In some embodiments, devices and methods described herein may be used to increase the strength of tissue healing at a location where a surgeon has created a bleb 127. In some embodiments, devices and methods described herein may be used to achieve increased permeability of a bleb 127. In some example embodiments, aqueous humor may pass through the wall of a bleb 127 and flow along the outer surface of the eye in a manner analogous to the flow of tears.

In the example embodiment of FIG. 14, a distal portion 103 of a catheter 100 is positioned near the bleb 127 formed in the eye 20. The example therapy method illustrated in FIG. 14 includes delivering a flow of photosensitizing fluid 109 to a target region 155 of the tissue and irradiating the target region with photo-activating light. In the stylized diagram of FIG. 14, photo-activating light emitted from a distal portion 103 of the catheter 100 is illustrated using solid triangles. The flow of photosensitizing fluid 109 exiting a distal portion 103 of the catheter 100 is illustrated using solid circles in the stylized diagram of FIG. 14. In some example embodiments, the photosensitizing fluid 109 comprises riboflavin. In some example embodiments, the photosensitizing fluid 109 comprises oxygen. In some embodiments, devices and methods described herein provide controlled, targeted, collagen crosslinking in tissue to improve biomechanical characteristics of the tissue. In some embodiments, devices and methods described herein strengthen tissue. In some embodiments, the strengthening of the tissue reduces stretch signals to fibroblasts. In some embodiments, the strengthening of the tissue reduces the likelihood that fibroblasts will transform into myofibroblasts. In some embodiments, devices and methods described herein inhibit excessive fibrosis. In some embodiments, devices and methods described herein fix tissues in place. In some embodiments, devices and methods described herein fix tissues in a way that prevents stretching of tissues during the healing phase. In some embodiments, devices and methods described herein fix tissues in a way that inhibits the “stretch” signal that promotes fibroblasts to transform into myofibroblasts.

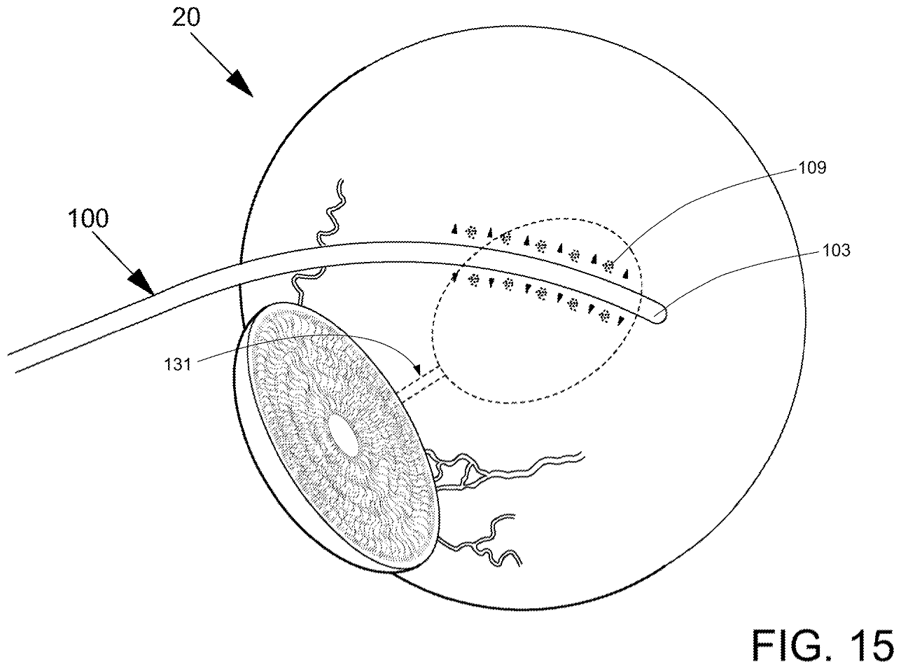

FIG. 15 is a stylized diagram illustrating an example method of treatment performed on an eye 20. With reference to FIG. 15, it will be appreciated that an aqueous humor drainage device has been implanted in the eye 20. In some example methods, glaucoma may be treated by implanting one or more aqueous humor drainage devices 131 in the eye. In some cases, the aqueous humor drainage device 131 may provide a pathway that allows aqueous humor to flow out of the anterior chamber of the eye. The aqueous humor drainage device 131 shown in FIG. 15 may be located, for example, under the episcleral of the eye 20.

An example aqueous humor drainage device 131 is illustrated using dashed lines in FIG. 15. Example methods in accordance with this detailed description may include implanting a device in the eye of a patient. Examples of devices that may be suitable in some applications include tube shunts (e.g., the PRESERFLO MicroShunt marketed by Santen Pharmaceutical Co. Ltd.), glaucoma implants (e.g., the BAERVELDT Glaucoma Implant marketed by Johnson and Johnson) and other devices used to treat glaucoma (e.g. the Ahmed valve). In some cases, the aqueous humor drainage device 131 may provide a pathway that allows aqueous humor to flow into a reservoir under the episcleral of the eye. In the example embodiment of FIG. 15, the distal end of a catheter is positioned near the aqueous humor drainage device 131. In some embodiments, devices and methods described herein inhibit excessive fibrosis. In some embodiments, devices and methods described herein provide lower postoperative intraocular pressure by performing collagen crosslinking in tissue to limit fibroblast proliferation and fibroblast migration. In some embodiments, devices and methods described herein provide lower postoperative intraocular pressure by performing collagen crosslinking in tissue to prevent excessive fibrosis. In some embodiments, devices and methods described herein provide lower postoperative intraocular pressure by performing collagen crosslinking in tissue to prevent scar formation.

The example therapy method illustrated in FIG. 15 includes delivering a flow of photosensitizing fluid 109 to tissues located near the aqueous humor drainage device 131 and irradiating those tissues with photo-activating light. In the stylized diagram of FIG. 15, photo-activating light emitted from the distal end 103 of the catheter 100 is illustrated using solid triangles. The flow of photosensitizing fluid 109 exiting the distal end 103 of the catheter 100 is illustrated using solid circles in the stylized diagram of FIG. 15. In some embodiments, devices and methods described herein prevent migration of an implant by performing episcleral and/or subconjunctival collagen crosslinking in the tissues located near the implant. In some embodiments, devices and methods described herein prevent erosion of tissues located near an implant by performing episcleral and/or subconjunctival collagen crosslinking in the tissue. In some embodiments, devices and methods described herein prevent implant exposure by performing episcleral and/or subconjunctival collagen crosslinking in the tissues located near the implant.

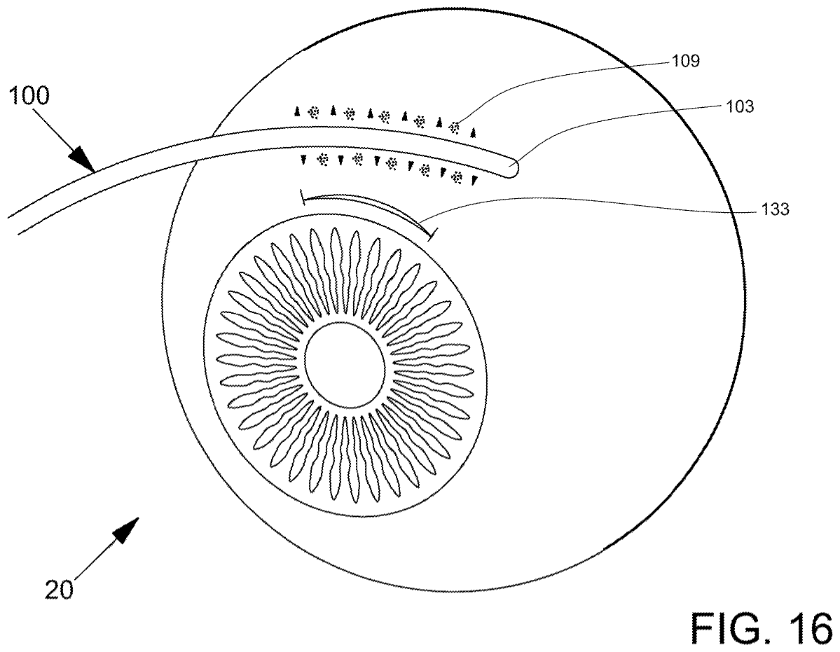

FIG. 16 is a stylized diagram showing an eye 20. With reference to FIG. 16, it will be appreciated that an incision 133 has been made in the tissue of the eye 20. In some example methods, a treatment procedure may include making incisions 133 using a scalpel. Methods and apparatus in accordance with this detailed description may be used to promote collagen crosslinking across incisions in some example embodiments. In the example embodiment of FIG. 16, a distal portion 103 of a catheter 100 is located near the incision 133 in the tissue of the eye 20. The example therapy method illustrated in FIG. 16 may include delivering a flow of photosensitizing fluid 109 to a target region of the tissue and irradiating the target region with photo-activating light. In the stylized diagram of FIG. 16, photo-activating light emitted from the distal end 103 of the catheter is illustrated using solid triangles. The flow of photosensitizing fluid 109 exiting the distal end 103 of the catheter 100 illustrated using solid circles in the stylized diagram of FIG. 16. In some example embodiments, the photosensitizing fluid 109 comprises riboflavin. In some example embodiments, the photosensitizing fluid comprises oxygen. In some embodiments, devices and methods described herein provide controlled, targeted, collagen crosslinking in tissue to improve biomechanical characteristics of the tissue. In some embodiments, devices and methods described herein strengthen tissue. In some embodiments, devices and methods described herein inhibit excessive fibrosis. In some embodiments, devices and methods described herein prevent scar formation. In some embodiments, devices and methods described herein fix tissues in a way that prevents stretching of tissues during the healing phase.

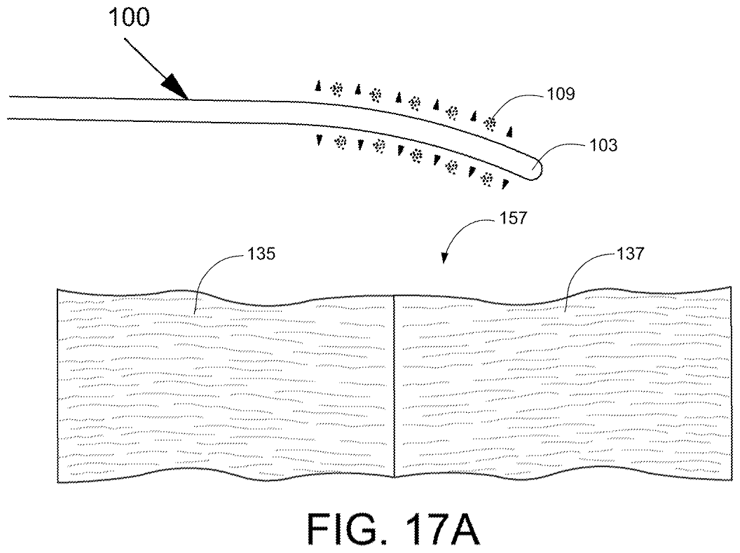

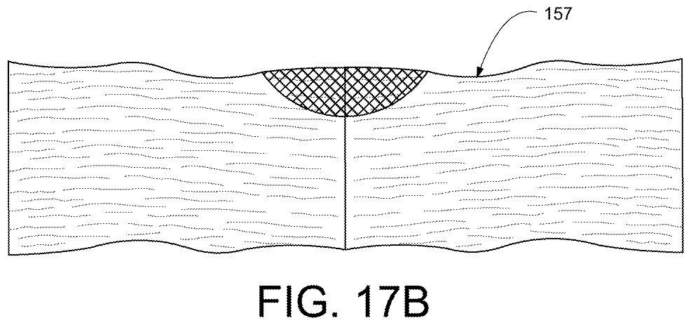

FIG. 17A and FIG. 17B are stylized diagrams illustrating a medical procedure in accordance with this detailed description. In the example embodiment of FIG. 17A, an incision has divided a tissue into a first portion 135 and a second portion 137. In some example methods, a treatment procedure may include making incisions using a scalpel. With reference to FIG. 17A, it will be appreciated that the distal end 103 of a catheter 100 has been positioned near the incision in the tissue in the embodiment of FIG. 17A. In some example methods, a catheter may be used to promote collagen crosslinking across incisions in tissue to promote healing of the tissue. The example therapy method illustrated in FIG. 17A includes delivering a flow of photosensitizing fluid 109 to a target region 157 of the tissue and irradiating the target region with photo-activating light. In the stylized diagram of FIG. 17A, photo-activating light emitted from the distal end 103 of the catheter 100 is illustrated using solid triangles. The flow of photosensitizing fluid 109 exiting the distal end 103 of the catheter illustrated using solid circles in the stylized diagram of FIG. 17A. In some example embodiments, the photosensitizing fluid 109 comprises riboflavin. In some example embodiments, the photosensitizing fluid 109 comprises oxygen.

FIG. 17B is a stylized diagram showing the tissue seen in FIG. 17A after the catheter has been used to promote collagen crosslinking across incisions in tissue to promote healing of the tissue. The collagen crosslinking is illustrated using a pattern of crosshatched lines in FIG. 17B. The medical procedure illustrated in FIG. 17A and FIG. 17B may be used to treat various body tissues and may be used in various areas of medicine. By way of example and not limitations, the devices and methods described herein may be used for applications in ophthalmology, orthopedics, urology, plastic surgery, general surgery, thoracic surgery, and cardiology. Examples of applications in ophthalmology include treatment of glaucoma (e.g., improved wound healing for glaucoma drainage devices), treatment of the retina (e.g., treatment to prevent erosion of scleral implants), scleral crosslinking to prevent myopia progression, and oculoplastics (e.g., less invasive ptosis repair and blepharoplasty). Examples of orthopedic applications include hip cartilage resurfacing prior to hip replacement, knee cartilage resurfacing prior to knee replacement surgery, and strengthening fibrous cartilage around collagen discs to contain disc herniation.

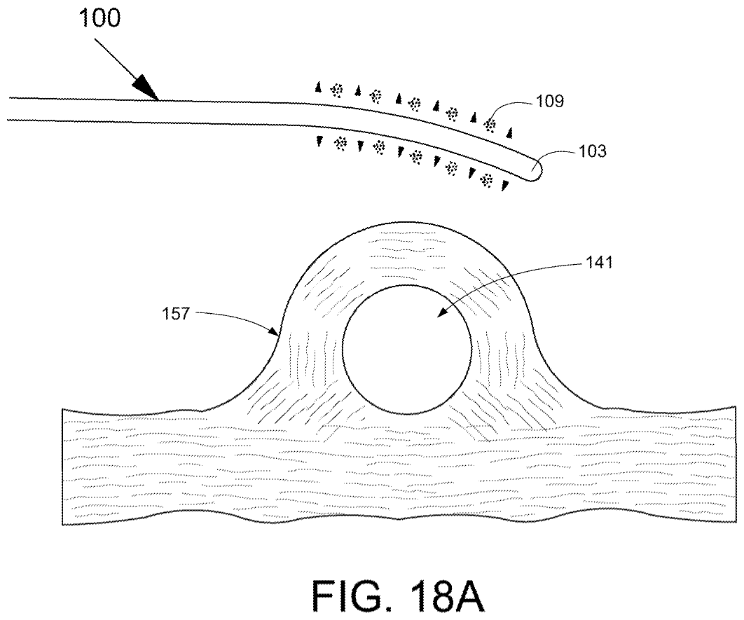

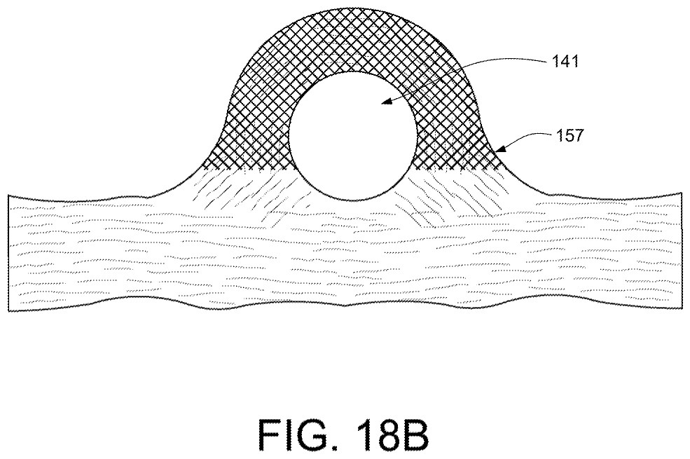

FIG. 18A and FIG. 18B are stylized diagrams illustrating a medical procedure in accordance with this detailed description. The stylized diagram of FIG. 18A includes a cross-sectional view of a region 157 of tissue defining a fluid flow channel 141. In some embodiments, devices and methods described herein may be used to reshape tissues into a desired configuration. In some embodiments, devices and methods described herein form tissue structures defining fluid flow channels 141 through the tissue. In some embodiments, devices and methods described herein form tissue structures that improve the transmission of aqueous humor in the eye 20 of a patient. With reference to FIG. 18A, it will be appreciated that the distal end of a catheter 100 has been positioned near the flow channel defined by the tissue in the embodiment of FIG. 18A. The example therapy method illustrated in FIG. 18A includes delivering a flow of photosensitizing fluid 109 to a target region of the tissue and irradiating the target region with photo-activating light 109. In the stylized diagram of FIG. 18A, photo-activating light emitted from the distal end 103 of the catheter 100 is illustrated using solid triangles. The flow of photosensitizing fluid exiting the distal end of the catheter illustrated using solid circles in the stylized diagram of FIG. 18A. FIG. 18B is a stylized diagram showing the tissue seen in FIG. 18A after the catheter 100 has been used to promote collagen crosslinking in the tissue. The collagen crosslinking is illustrated using a pattern of crosshatched lines in FIG. 18B.

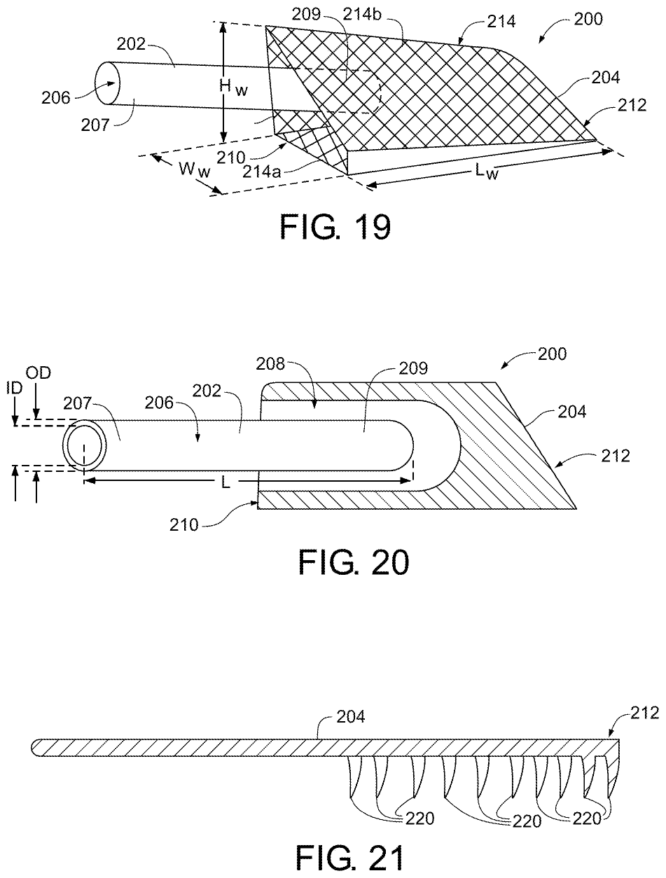

Referring to FIGS. 19-27, embodiments of an aqueous humor wicking device 200 and its applications are depicted. The aqueous wicking device 200 may be used in conjunction with treatment methods described above with respect to FIGS. 1-18, or may be used independently, and as described further below with respect to the figures. The crosslinking treatment methods described above work most effectively with the aqueous wicking device 200 because it optimizes wound healing through multiple mechanisms to deliver the best postoperative outcome in terms of ideal postoperative intraocular pressure (IOP) as well as longevity of the surgical procedure. Performing glaucoma surgery using the crosslinking procedure alone may either have a higher than desired postoperative IOP because it does not have the increased surface area of outflow provided by the aqueous wicking device. Furthermore, without the microfilter membrane, proinflammatory mediators from the aqueous can still reach tissues beyond the glaucoma drainage device, cause increased scarring and a suboptimal IOP. Also, the woven design of the aqueous wicking device allows for the same amount of draining with a smaller footprint. A smaller footprint requires a smaller surgical dissection, which causes less trauma and tissue inflammation and results in improved surgical outcomes. The combined innovations of the crosslinking treatment methods, with microfilter membrane, and woven design will result in the optimal surgical outcomes and are all necessary components.

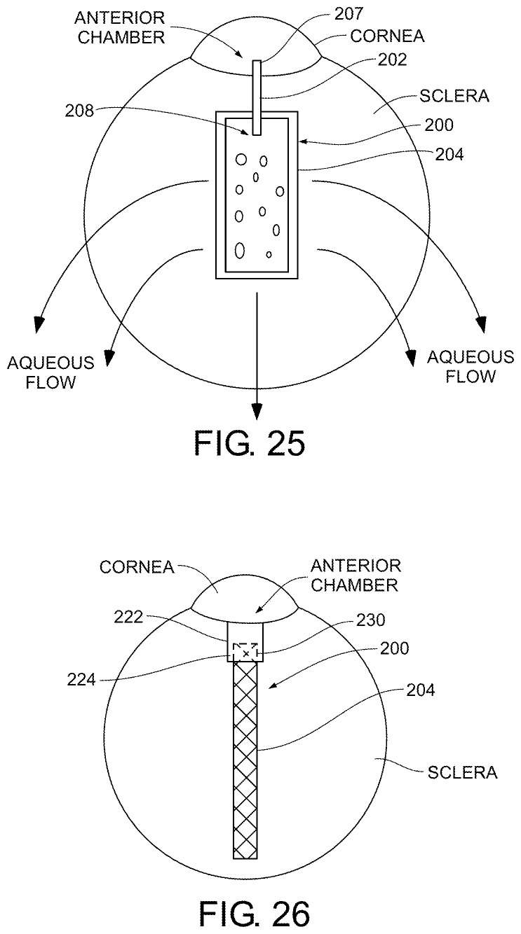

Referring specifically to FIGS. 19 and 20, aqueous wicking device 200 is depicted. In this embodiment, aqueous wicking device 200 includes tube 202 coupled with wicking portion 204, which in an embodiment forms a woven wicking reservoir. In another embodiment described below with respect to FIGS. 25-27, aqueous wicking device 200 may not include a tube 202.

Still referring to FIGS. 19 and 20, tube 202 defines flow channel 206 and has length L, outside diameter OD and inside diameter ID. Dimensions of length L may vary depending on a number of factors, including the dimensions of the particular wicking portion 204, tube 202 and wicking portion 204 placement, and so on. Dimension of outside diameter OD and inside diameter ID may also vary depending on a number of factors, such as desired fluid flow through tube 202, wicking or capillary or capillary action properties of wicking portion 204, and so on. In an embodiment, tube 202 comprises a shunt or more particularly, a microshunt.

Tube 202 may be considered a resistive flow element in that the inside diameter and length of tube 202 present a resistance to the amount of aqueous that may flow out of the anterior chamber in response to capillary action produced by wicking portion 204, as described further below.

Tube 202 defines anterior end 207 and posterior end 209. Anterior end 207 is configured to interface directly with tissue and aqueous of the eye, as described further below, while posterior end 209 is configured to interface, or couple with, wicking portion 204.

Wicking portion 204, in an embodiment, —includes anterior end 210 and posterior end 212. Wicking portion 204 also includes exterior walls or wall structure 214. Walls 214 may comprise a single, integral cylindrical wall, or may comprise multiple walls, such as those depicted, e.g., a first or interior or bottom wall 214a, a second or exterior or top wall 214b, and a third or side wall. In the case of a square or rectangular cross section, an additional or fourth wall 214 may be included.

In an embodiment, wicking portion 204 comprises a biocompatible woven material capable of creating a capillary action, for example a polypropylene material used for FDA-approved glaucoma drainage implants. Such material may include Ahmed Implants S2,S3,B1,PS2,PS3; Molteno Implants S1,D1,M1,R2/L2;DR2/DL2,GS,GL0. Materials may also include Wicking Polytetrafluoroethylene (PTFE)/Expanded Polytetrafluorothylene ePTFE. Other examples of materials may be hydrogels. Embodiments of the invention are not limited to these particular examples, and may include other wicking materials capable of providing the desired capillary action required for the described devices and treatments herein.

Due to its wicking properties, wicking portion 204 functions as a liquid reservoir, retaining aqueous. In an embodiment, wicking portion 204 may define an optional reservoir cavity 208 formed by walls 214. Reservoir cavity 208 may initially comprise an air pocket that after implantation may be partially or fully filled with aqueous, so as to increase the volumetric reservoir capacity of wicking portion 204. In an embodiment, reservoir cavity 208 may initially be present before implantation, but after implantation, may diminished in size, or even completely removed, due to compression of wicking portion 204 upon implantation.

In embodiments, the material of wicking portion 204 is a flexible material which reduces the risk of device erosion.

Further, the use of woven materials increases surface area and aqueous transmission via increased capillary action. This potentially allows significant aqueous transmission with a relatively small device footprint.

In an embodiment, wicking portion 204 consists of a single material, which may be a woven material, such as one described above. In other embodiments, wicking portion 204 may comprise a composite material, i.e., more than one material or layer. In one such embodiment, which is described below in further detail, wicking portion 204 includes a multi-weave material that includes one or more layers of a coarse-weave material and one or more layers of a fine-weave material.

As depicted in FIG. 19, wicking portion 204 defines a length Lw, width Ww and height Hw. In an embodiment length Lw is generally greater than height Hw and width Ww such that wicking portion 204 is strip-like in character. As described below, wicking portion 204 may define other shapes. In an embodiment, height Hw is relatively small with respect to the eye, so that wicking portion 204 maintains an ultra-low profile. In an embodiment, height Hw is in a range of 0.1 mm to 0.6 mm. In one particular embodiment, height Hw is no greater than 0.4 mm. Having a low profile, or small height Hw, minimizes the stretch response for fibroblasts, since the tissues are not as stretched as much accommodate and cover the implant. Reducing the amount of stretch on the tissues reduces the signal to mechanoreceptors on the fibroblasts. Therefore this results in a reduced rate of transformation of fibroblasts to myofibroblasts.

In an embodiment, wicking portion 204 may comprise a single, integral structure, such as a single posterior strip. In other embodiments, wicking portion 204 may comprise multiple connected parts, such as a central posterior strip with multiple radiating strips, as described further below with respect to FIG. 28.

In an embodiment, wicking portion 204 may define a generally flat and low profile cross section when viewing an end of the wicking portion. In other embodiments, wicking portion 204 may define other shapes in cross section, such as a square or rectangular shape.

Referring specifically to FIG. 20, aqueous wicking device 200 is depicted in cross section. As depicted, posterior end 209 is inserted into wicking portion 204, and terminates in reservoir cavity 208. Anterior end 207 terminates outside and away from wicking portion 204. Anterior end 207 is in fluid communication with posterior end 209, and connected by fluid channel 206 of tube 202.

At the region of wicking portion 204 where a portion of tube 202 enters wicking portion 204, a biocompatible adhesive or sealant may be used to retain placement of tube 202 at wicking portion 204. In other embodiments, tube 202 is coupled to wicking portion 204 simply by a friction fit.

In an embodiment, and as depicted, reservoir cavity 208 is generally larger than the portion of tube 202 that is inserted into the reservoir cavity, thereby creating a space around tube 202. As described above, reservoir cavity 208 may be present prior to implantation, which allows for easier insertion of tube 202 into wicking portion 204. However, reservoir cavity 208 may collapse fully or partially around tube 202 after implantation. In embodiment wherein reservoir cavity 208 is not fully collapsed after implantation, aqueous will enter the reservoir cavity 208 space prior to being transmitted through walls 214, and the interstitial space about tube 202 allows newly-entering aqueous to be distributed within the cavity for eventual flow to the exterior of wicking portion 204. In an embodiment wherein reservoir cavity 208 is fully or substantially collapsed, aqueous exits 202 and is drawn directly into wicking portion 204.

Although somewhat flexible and to a certain degree compressible, the structure of wicking portion 204 provides a physical support to tube 202 so as to prevent fibrosis from sealing off the outlet of the tube. In any case, both tube 202 and wicking portion 204 may be configured to be periodically replaced over the lifetime of the patient.

The size and shape of reservoir cavity 208 may vary depending on whether it functions primarily as just a tube cavity for receiving tube 202, or whether it is intended to include additional space to hold aqueous. In an embodiment, cavity 208 may the same or very close to the same size as that portion of tube 202 within cavity 208 such that tube 202 fits relatively tightly within wicking portion 204. Such an embodiment may be useful in creating a lower profile device. In such an embodiment, when tube 202 is inserted into wicking portion 204, the space of cavity 208 is filled, or substantially filled, with tube 202.

In an alternate embodiment, a length of cavity 208 is longer than the portion of tube 202 within cavity 208, and a circumference of cavity 208 is larger than the outside diameter of tube 202, so as to allow aqueous to enter cavity 208 and flow about the interior space of cavity 208 prior to transmission through the weave material.

In an embodiment, superficial portions of wicking portion 204 may include a capsule or fibrosis marker. In an embodiment such a marker may comprise a blue color or other readily visible color. As the capsule around aqueous wicking device 204 heals, it is possible to assess the amount of light reflectivity and correlate that to the amount of scar tissue present, thereby informing postoperative care, which could include treatment with more steroids, NSAIDS, 5FU, MMC, MMP inhibitors, etc.

Referring to FIG. 21, in an embodiment, aqueous wicking device 200 or wicking portion 204 may include a plurality of bio-barbs 220, including at posterior end 212. Barbs 220 may extend transversely from wicking portion 204 as depicted, and may include relatively pointed or sharp end portions configured to pierce or otherwise grip tissue of the eye, e.g., the sclera, allowing scleral fixation of device 200 to the eye without the need for sutures.

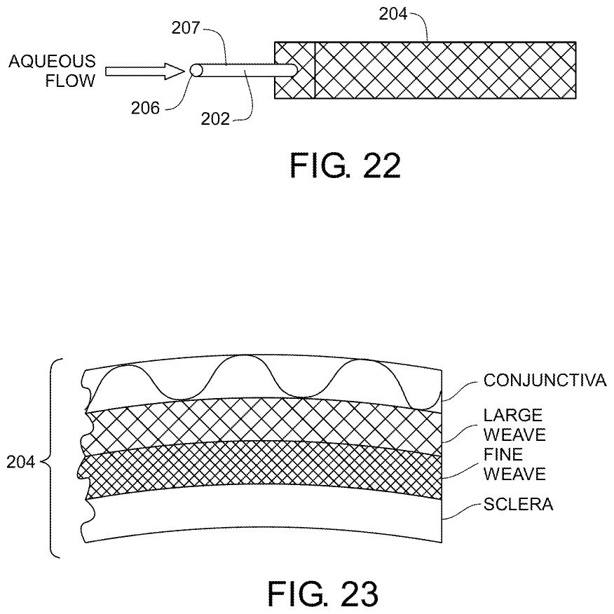

Referring to FIG. 22, and as explained further below, after implantation into the eye, the capillary action of wicking portion 204 will draw aqueous from the anterior chamber of the eye into anterior end 207 of tube 202, through tube channel 206 and into wicking portion 204, followed by filtering of the aqueous and transmission of the aqueous to other areas of the eye.

Referring to FIG. 23, a multi-weave embodiment of a portion of wicking portion 204 is depicted as implanted between the conjunctiva and sclera of the eye. A less dense weave structure allows more structural support to prevent the wicking portion 204 from collapsing as the conjunctival heals and contracts overtop. The dense weave structure allows for more surface area to enhance aqueous drainage.

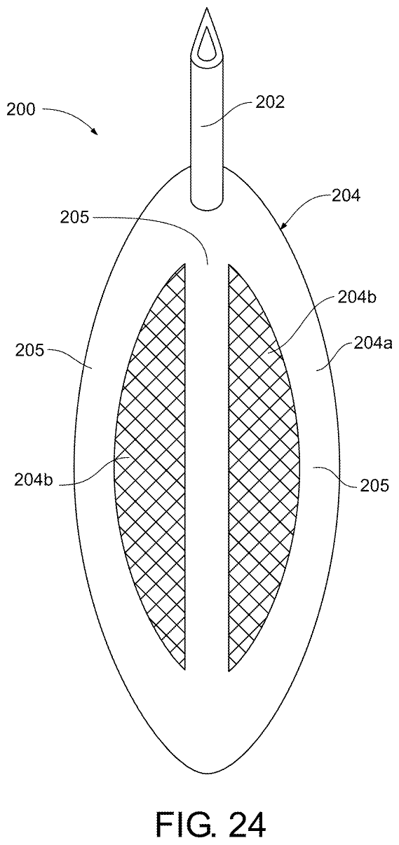

An embodiment of a multi-weave aqueous wicking device 200 is depicted in FIG. 24. Similar to the other embodiments described herein, aqueous wicking device 200 comprises tube 202 and wicking portion 204. In this embodiment, wicking portion 204 is a multi-weave-type device that includes thick weave, large-diameter fiber portion 204a, and fine weave, small-diameter fiber portions 204b. The thicker weaver and larger fibers of 204a provide structural support to prevent contracting tissues from collapsing the finer weave portions 204b. The finer weave of smaller fibers, 204b, provides more surface area for maximum aqueous absorption.

In an embodiment, and as depicted, thick weave, large-diameter fiber portion 204a may include multiple strands 205 extending longitudinally about an exterior portion of wicking portion 204. In other embodiments, portion 204a may also include, or alternatively comprise, latitudinally-extending strands 205. In an embodiment, thick weave, large-diameter fiber portion 204a may include a strand 205 underneath some of fine weave, small-diameter fiber portions 204b so as to provide additional structure support to wicking portion 204.

Methods of the invention include making an incision and creating a capsule between the conjunctive and sclera, and inserting wicking portion 204 and all or a portion of tube 202 into the capsule created. Areas underneath the conjunctival/Tenon's incision are watertight to reduce risk or early wound leakage.

In this particular embodiment, wicking portion 204 is a multi-weave embodiment that includes an exterior large-weave portion and in interior fine-weave portion, though it will be understood that a single-weave material embodiment of wicking portion 204 may also be used, and also implanted between the conjunctive and sclera.

With the multi-weave, multi-layer embodiment depicted, the fine weave located closer to the interior of the eye may have a tighter weave of smaller fibers to as to create a stronger capillary action, or a higher surface area to aqueous movement. Further making a superficial or outer portion of wicking portion 204 of relatively larger woven fibers provides additional structural strength and prevents unwanted compression of wicking portion 204. This can be important because as the conjunctiva heals around the glaucoma device, the conjunctival tissues contract. Without the woven material, the healing conjunctiva would seal off the outflow portion of the tube, and the surgery would fail.

Methods of the invention described herein include implanting aqueous wicking device 200 in the human eye. For example, referring specifically to FIG. 25, an embodiment of aqueous wicking device 200 implanted in an eye is depicted, with the conjunctiva of the eye not shown for illustrative purposes.

A method for implanting aqueous wicking device 200 includes the following steps. Either a fornix-based or limbal-based incision is used to dissect conjunctiva and Tenon's from sclera. The aqueous wicking device is fixated onto the sclera approximately 2-4 mm posterior to the limbus. Access to the anterior chamber is created through a number of methods. A needle/blade ranging from approximate caliber 30G-20G tunnels through the sclera and enters the anterior chamber. The wicking device is inserted into this tunnel and this tunnel fixates the wicking device to the sclera. Conjunctiva/Tenon's is further dissected as posteriorly as possible. The distal portion of the wicking device is placed in this space and the wicking device is secured to the sclera with barbs 220 to contact, and possibly pierce, portions of the sclera. The second method for the wicking device to access the anterior chamber is to fashion a trabeculectomy-style scleral flap, approximately 3 mm×3 mm, and creating an ostomy between the anterior chamber and the subconjunctival space. The scleral flap is tied down to restrict flow with 10-0 nylon sutures. The anterior portion of the wicking device can be implanted either within the scleral flap, or just posterior to the scleral flap. The third option is to insert the anterior end 210 of tube 202 through the incision and into the anterior chamber, and having the aqueous flow into the aqueous wicking device.

Still referring to FIG. 25, aqueous wicking device 200 is depicted in cross section as implanted in the eye. As described above, after implantation, anterior end 207 of tube 202 is positioned in the anterior chamber, and thusly in fluid communication with the aqueous humor or aqueous in the anterior chamber of the eye. In an embodiment, an end of wicking portion 204 is placed approximately 5 mm posterior to the limbus. The capillary action of wicking portion 204 draws aqueous out of the anterior chamber, through tube 202 and into the wicking portion 204, including into cavity 208, where the aqueous is filtered by the wicking portion 204 and released to other portions of the eye, including the conjunctiva and sclera.

Wicking portion 204 functions as a replaceable microfilter membrane, sequestering proinflammatory cytokines and proteins/factors from the aqueous so that that the fluid that reaches the conjunctiva/Tenon's/Episclera is minimally pro-inflammatory. In other words, the filtered aqueous is similar to a balanced salt solution. This reduces the amount of scarring around the capsule that surrounds the reservoir, or wicking portion 204, and also ensures a stable postoperative IOP in the target range. Another embodiment is that the woven material itself acts as a sink to sequester proinflammatory mediators and cytokines for a period (lasting hours to weeks) that allow the cytokines to break down before having a chance to interact with the surrounding tissues and induce excessive scarring. The woven material could be composed of or coated with hydrogels, which are materials that trap/sequester cytokines.

Further, wicking portion 204 with its filtering function can also serve as a depot for slow release medications or therapeutics that modulate wound healing, inhibit fibrosis and fibroblast transdifferentiation into myofibroblasts. Other slow release medications can also control vascular tone of the post trabecular outflow system (i.e. episcleral venous tone) and enhance uveoscleral outflow of aqueous.

Wicking portion 204 can be surgically accessed and replaced numerous times of the course of the patient's lifetime.

Referring to FIGS. 26-28, another embodiment of aqueous wicking system 200 is depicted. System 200 as depicted in FIGS. 26-28 is substantially similar to the embodiments described above, with the primary exception being that wicking portion 204 is in direct fluid communication with the anterior chamber, rather than a tube 202. In other words, in the embodiments of FIGS. 26-28, aqueous wicking system 200 does not include tube 202, and instead, relies on an end of wicking portion 204 to function as the flow restriction component.

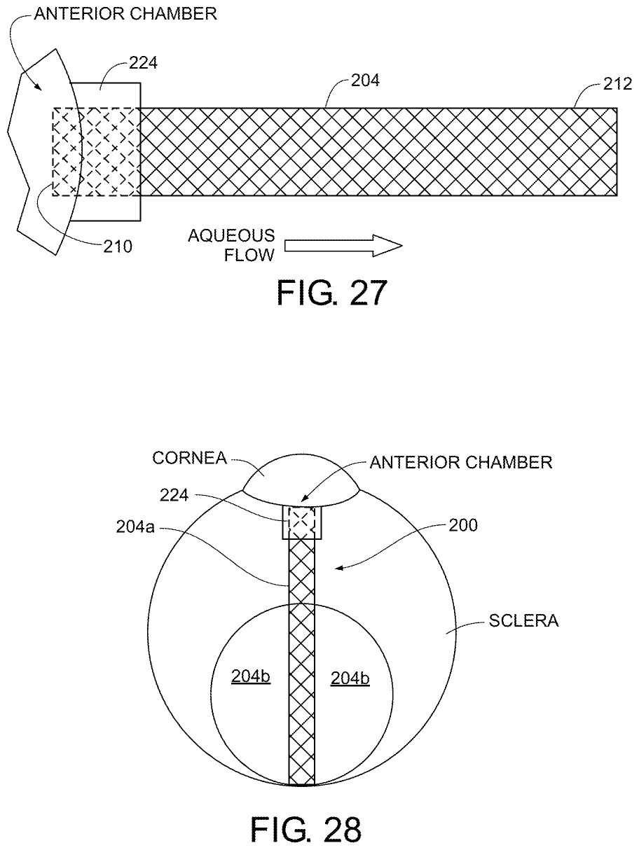

Referring specifically to FIGS. 26 and 27, an embodiment of a tubeless version of aqueous wicking system 200 is depicted. Wicking portion 204 may be substantially described above with respect to FIGS. 19-23. In this embodiment, incision 222, which may be a fornix- or limbal-based incision, is made to fashion a scleral flap 224. An ostomy is made, anterior end 210 of wicking portion 204 is inserted into the anterior chamber of the eye. The remaining portion, including distal portion 212 of wicking portion 204 is implanted as described above between the conjunctiva and sclera. Flow restriction is performed by suturing the scleral flap to the scleral bed.

Aqueous flow volume is determined in part by wicking properties of the size and cross section of that portion of wicking portion 204 that is inserted into the anterior chamber.

An advantage of this arrangement is that it creates an artificial trabecular meshwork or filter that is unlikely to be obstructed by blood and/or fibrin. As such this embodiment may present some advantages over an embodiment employing a microshunt or tube 202. Further, since only a small volume of wicking portion 204 need be inserted into the anterior chamber to produce a large amount of surface area, wicking portion 204 need only be placed a short distance into the anterior chamber and thereby kept away from the endothelium and iris.

Referring now to FIG. 28, an alternate embodiment of implantable aqueous wicking device 200 is depicted. As briefly described above, wicking portion 204 may comprise various shapes and configurations. In the embodiment depicted in FIG. 28, wicking portion 204 includes central strip 204a as well as a pair of auxiliary wicking portions 204b. Auxiliary wicking portions 204b as depicted are coupled to central strip 204a and extend transversely from strip 204a and add additional wicking capacity to wicking portion 204. In an embodiment, portions 204b may comprise the same material and capacity as central strip 204a, but in other embodiments, may comprise a different material that has more or less wicking capacity, i.e., capillary action. In this embodiment, the capillary action of wicking portion 204 may be increased incrementally by adding various sizes of auxiliary portions 204b.

As described above, with respect to FIGS. 19-28, the multiple embodiments of wicking device 200 create a capillary action to draw aqueous from the anterior chamber of the eye, followed by filtration by wicking portion 204 prior to release into other areas of the eye, including the conjunctiva and sclera. Wicking portion 204 can sequester or remove pro-inflammatory cytokines and proteins. As described, the woven materials, including fibers, of wicking portion 204 act as a sink to sequester pro-inflammatory mediators and cytokines, allowing the cytokines to break down in, on, or within wicking portion 204. The use of hydrogels may aid in capturing cytokines.

As described above, embodiments of wicking portion 204 may be sorbent, absorptive and/or adsorptive. With an absorptive wicking portion 204, the aqueous is substantially soaked up or absorbed into wicking portion 204, which may include absorption of aqueous by materials and coatings of wicking portion 204, including its woven fibers. This absorption effect not only creates the capillary effect, but also aids in the sequestering of pro-inflammatory cytokines.

Embodiments of wicking device 200, including those described above with respect to FIGS. 19-28 may be configured to specifically include adsorptive properties, in addition to, or in some embodiments, rather than, the absorptive properties described above, so as to further enhance the removal of certain cytokines from the aqueous. As one of ordinary skill will understand, in an adsorption process, atoms, ions or molecules of the aqueous are retained onto a surface of an adsorbent.

Glaucoma patients with high IOPs may also have significant amounts of pro-inflammatory cytokines in their aqueous, as compared to patients without glaucoma, as described in “Pro-Inflammatory Cytokines in Glaucomatous Aqueous and Encysted Molteno Implant Blebs and Their Relationship to Pressure”, Jeffrey Freedman and Pavel Iserovich as published July 2013, in Volume 54, Issue 7 of Investigative Ophthalmology & Visual Science, which is incorporated herein by reference in its entirety. Certain cytokines may cause changes in the cells of the trabecular meshwork, resulting in a decrease in aqueous outflow, thereby causing an elevation in IOP. The removal of such pro-inflammatory cytokines may reduce IOP as part of a glaucoma treatment.

According to embodiments of the invention, aqueous filtering or purification using adsorption may be particularly effective in removing or sequestering cytokines and so reducing IOP and treating glaucoma. Such embodiments, may include adsorptive wicking devices 200, or other devices and techniques adapted to utilize adsorption to remove or sequester cytokines.

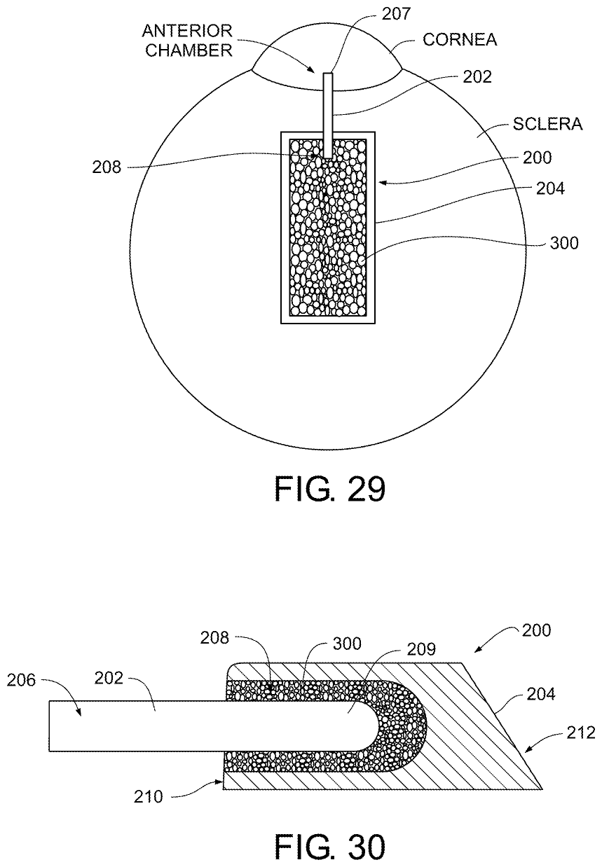

Referring generally to FIGS. 29-32, wicking device 200 may be configured to include cytokine adsorpters, such that wicking device 200 comprises an implantable adsorptive filtering device for aqueous purification and glaucoma filtration surgery.

Referring specifically to FIG. 29, depicted wicking device 200 is substantially the same as wicking device 200 depicted in FIG. 25, which depicts a sectional view of wicking device 200 implanted into an eye, with the exception that the wicking device 200 of FIG. 29 includes a plurality of cytokine-adsorptive micro-beads 300. Similar to the embodiment of FIG. 25, wicking device 200 of FIG. 29 includes tube 202 with anterior end 207 in communication with the anterior chamber of the eye. Wicking portion 204 defines cavity 208, which receives posterior end 209, thereby connecting the aqueous-filled anterior chamber with cavity 208, such that aqueous flows from the anterior chamber into cavity 208 via tube 202 due to the pressure in the anterior chamber and the capillary forces created by wicking portion 204.

In this embodiment, cavity 208 includes the plurality of cytokine-adsorptive micro-beads 300. As depicted, cavity 208 may be entirely or substantially filled with cytokine-adsorptive-micro-beads in a tightly-packed pattern. In such an embodiment, aqueous will fill the spaces around cytokine-adsorptive micro-beads 300. In other embodiments, cavity 208 may not be substantially filled with cytokine-adsorptive micro-beads 300, so as to leave a larger volume to be filled with aqueous, thereby accommodating a relatively higher flow of aqueous.

After implantation, aqueous flows into cavity 208, bringing aqueous into contact with surfaces of cytokine-adsorptive micro-beads 300. As described further below, cytokine-adsorptive micro-beads 300 will adhere to the surfaces of cytokine-adsorptive micro-beads 300, thereby removing the cytokines from the aqueous and retaining them within wicking device 200. The filtered aqueous flows out of wicking portion 204 to other parts of the eye, and cytokines remain within wicking device 200, adhered to surfaces of cytokine-adsorptive micro-beads 300.

Referring also to FIG. 30, another embodiment of a cytokine-adsorbent wicking device 200 that functions as an adsorptive filtering device, is depicted. In this embodiment, wicking device 200 is substantially similar to the embodiment of wicking device 200 of FIG. 19, but with the addition of cytokine-adsorptive micro-beads 300 in cavity 208 of wicking portion 204.

In an embodiment, cytokine-adsorptive micro-beads 300 comprise small beads that may be made of a biocompatible polymer material. In another embodiment, cytokine-adsorptive micro-beads 300 may comprise resins, or macro-porous resins. In an embodiment, cytokine-adsorptive micro-beads 300 are sized such that many, perhaps hundreds, of cytokine-adsorptive micro-beads 300 will fit in cavity 208. In an embodiment, cytokine-adsorptive micro-beads 300 are similar in size to a grain of salt, or approximately 0.3 mm in diameter. In an embodiment, a diameter of cytokine-adsorptive micro-beads 300 ranges from 0.2 mm to 0.4 mm. In other embodiments, cytokine-adsorptive micro-beads 300 are larger or smaller. In an embodiment, a diameter of beads 300 ranges from 0.3 mm to 1.5 mm; in another embodiment, a diameter of beads 300 ranges from 0.6 mm to 1.2 mm.

In an embodiment, the surface of the biocompatible cytokine-adsorptive micro-beads 300 may be porous, having pores sized to trap and retain smaller cytokines, which generally are hydrophobic, while allowing larger components such as red blood cells to pass between the beads. Pore size may be relative constant for each bead 300 so as to target a particular cytokine, or may vary in size for each bead 300 so each bead is capable of adhering to adhere to multiple sizes and types of cytokines.

In an embodiment, cytokine-adsorptive micro-beads 300 may be mixed with hydrogels in cavity 208 to enhance the capture of cytokines, many of which are known to be hydrophobic.

As described above, the removal or sequestering of pro-inflammatory cytokines may decrease inflammation and subsequently IOP. Embodiments of wicking device 200 may be configured to filter particular pro-inflammatory cytokines, such as CXLCL1, CCL2, CXCL5, 3, and 4; and TGF-02, through the use of specifically-configured beads using pore sizing and shaping, and particular coatings or hydrogels to trap and retain targeted cytokines.

In an embodiment, cytokine-adsorptive micro-beads 300 may comprise CytoSorb© beads, as produced by CytoSorbents Corporation of Monmouth Junction, N.J., USA, marketed for use in blood filtering.

In other embodiments, rather than utilizing cytokine-adsorptive micro-beads 300, the fibers of wicking portion 204 may be coated with adsorbent or hydrogel substances, or the fibers themselves may include pores of a size to retain cytokines.

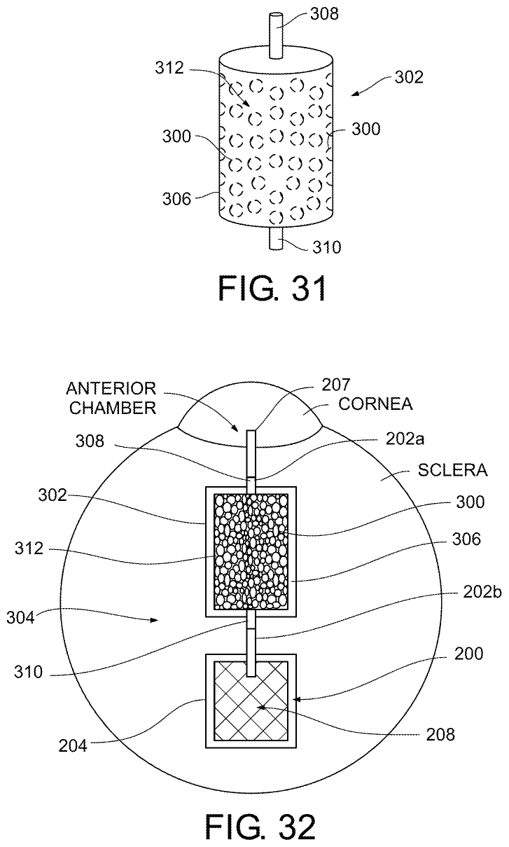

In the embodiments described above, cytokine-adsorptive micro-beads 300 may be inserted directly into wicking portions 204 of wicking device 200. However, in other embodiments, wicking device 200 may include a separate cytokine-adsorption device 302, as depicted in FIGS. 31-32, which may form aqueous filtering system 304 comprising wicking device 200 and cytokine-adsorption device 302.

Referring specifically to FIG. 31, an embodiment of cytokine-adsorption device 302 is depicted. In the depicted embodiment, cytokine-adsorption device 302 includes housing 306, inlet 308, outlet 310 and defines cavity 312. Cavity 312 includes a plurality of cytokine-adsorptive micro-beads 300 (dashed lines), having the composition and properties described above. In an embodiment, cytokine-adsorptive micro-beads 300 may be mixed in with a hydrogel in cavity 312.

In an embodiment, housing 306 may comprise a relatively rigid, though biocompatible structure, comprised of a polymer or resin material, and configured to be implanted in the eye. In such an embodiment, aqueous will flow in via inlet 308, and out via outlet 310. In such an embodiment, cytokine-adsorption device 302 may comprise an assembled biocaompatible cartridge. In an embodiment, housing 306 may form a generally cylindrical shape, though other shapes are contemplated.

In other embodiments, housing 306 may comprise a flexible material, similar to a sack or pouch, but still having an inlet 308 and an outlet 310. In one such embodiment, housing 306 may comprise a membranous material that allows some filtered aqueous or other molecules to flow out through the membrane that is housing 306, and to portions of the eye, such as the conjunctiva and sclera.

Referring also to FIG. 32, as depicted, inlet 308 may comprise a tube-like structure defining an inlet channel, and outlet 310 may also comprise a tube-like structure defining an outlet channel. Inlet 308 may be configured to attach to tube portion 202a, or may be long enough have an end that is directed inserted into the anterior chamber of the eye. Outlet 310 may be configured to attach to tube portion 202b of wicking device 200. In other embodiments, inlet 308 and outlet 310 may simply be openings in housing 306 configured to receive portions of tube 202, which may include tube portion 202a and 202b, respectively.

Referring specifically to FIG. 32, in an embodiment, cytokine-adsorption device 302 at its inlet is connected to tube portion 202a of tube 202, which is in fluid communication with the anterior chamber of the eye. Outlet 310 of cytokine-adsorption device 302 is connected to tube portion 202b, so as to be in fluid communication with wicking device 200, including cavity 208 of wicking portion 204.

In operation, the woven fibers of wicking portion 204 create capillary forces that draw aqueous from the anterior chamber, through tube portion 202a, and into cavity 312. The aqueous contacts surfaces of cytokine-adsorptive micro-beads 300, and through the adsorption process described above, binds cytokines to cytokine-adsorptive micro-beads 300, thusly filtering out cytokines in a first-stage filtering process.

Aqueous then flows out of cytokine-adsorption device 302, which may also be referred to as a first-stage filter or filtering device, through tube portion 202b, and into cavity 208 of wicking portion 204. Wicking portion 204 not only provides the capillary forces to draw the aqueous out of the anterior chamber and into cytokine-adsorption device 302 and wicking device 200, but also acts as a second-stage filtering device. The fibers of wicking portion 204 may further filter out and retain cytokines not trapped by beads 300, or may filter and retain types of cytokines not meant to be filtered out by beads 300. Wicking portion 204 and its cavity 312 may also include medications and/or therapeutics to be absorbed into the aqueous.

After being received by wicking device 200 and being subjected to second-stage filtering, the aqueous flows out of wicking portion 204 and into the eye, which may include flowing into the sclera of the eye.

While aqueous filtering system 304 may include both wicking device 200 and cytokine-adsorption device 302 in combination, or a adsorbent wicking device 200 with adsorptive beads or other adsorptive materials, cytokine-adsorption device 302 may also be used independently, without wicking device 200, as a stand-alone filtering device, or as part of another glaucoma treatment solution.

In addition to the embodiments of cytokine-adsorption device 302 described above, other embodiments may utilize adsorption to filter out, remove or sequester cytokines.

In one such embodiment, a cytokine-adsorption device 302 includes housing 306 in the form of a membrane, as described in part above. In one such embodiment, the membrane itself may be an adsorptive membrane. In one such an embodiment, membrane 306 may be a single or multi-layer membrane made of a polymer material. Some or all layers being adsorbent of pro-inflammatory cytokines. Adsorbtion of cytokines may be accomplished through the use of a combination of hydrophobic and hydrophilic materials to selectively bind either hydrophobic or hydrophilic cytokines.

In an embodiment, cytokine-adsorption device 302 comprising a membranous housing, or membrane 306, may be rolled or folded to be inserted through small incisions in the eye, independent of wicking device 200. Such membranous devices 302 may include adsorbent membranous layers and/or may include adsorbent materials, such as beads 300, within cavities formed by the membrane 306.

Cytokine-adsorption device 302, including membranous embodiments thereof, could be implanted for short-term use, and in some cases, after the effectiveness of the device 302 is past its peak, and after the device has degraded over time and with use, the device could be exchanged with a fresh membrane after a predetermined period of time. In other words, cytokine-adsorption device 302 could be a short-term temporary implant.

However, in other embodiments, cytokine-adsorption device 302 could be a permanent implant, particularly for embodiments having a longer-lasting housing 306.

In yet other embodiments, cytokine-adsorption device 302 could be replenished with adsorbent material, either through injection of new material, or by removal and exchange of adsorbent material.

In another embodiment, rather than cytokine-adsorptive micro-beads 300, cytokine-adsorption device 302 or wicking device 200, may include other types of materials, such as fibers or meshes having coatings.

In some embodiments, adsorbent materials, such as beads 300 or adsorbent resins could be injected directly into the subconjunctival space. In an embodiment, such adsorbent, cytokine-attracting resins would have sufficient plasticity to be biomechanically compatible with the surrounding tissue. A mixture of an injectable hydrogel with beads 300 could also be used. The hydrogel would prevent the migration of the beads into unwanted areas.

In other embodiments, aqueous filter of cytokines could be accomplished using electrochemical means. Another embodiment is that the adsorbent materials may slowly be absorbed overtime and may need to be periodically replenished with repeat injections of adsorbent materials into the subconjunctival space.

This application also applies to the concept of using the above technologies to purify cytokines from bodily fluids/organs within the human body, such as synovial fluid within joints in orthopedic and interstitial fluid during wound healing. This approach could also potentially be used in oncology for treating areas of smoldering inflammation to prevent tumorigenesis. Also, this technology could be injected within and or surrounding tumor around the time of radiotherapy/immunotherapy/chemotherapy to reduce the risk of cytokine storm during oncology treatment and surgery.

The following United States patents are hereby incorporated by reference herein: U.S. Pat. Nos. U.S. Pat. Nos. 9,113,843, 9,220,834, 9,241,641, 9,333,293, 9,352,105, and 9,457,140. The above references to U.S. patents in all sections of this application are herein incorporated by references in their entirety for all purposes. Components illustrated in such patents may be utilized with embodiments herein. Incorporation by reference is discussed, for example, in MPEP section 2163.07(B).

All of the features disclosed in this specification (including the references incorporated by reference, including any accompanying claims, abstract and drawings), and/or all of the steps of any method or process so disclosed, may be combined in any combination, except combinations where at least some of such features and/or steps are mutually exclusive.

Each feature disclosed in this specification (including references incorporated by reference, any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any incorporated by reference references, any accompanying claims, abstract and drawings), or to any novel one, or any novel combination, of the steps of any method or process so disclosed The above references in all sections of this application are herein incorporated by references in their entirety for all purposes.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents, as well as the following illustrative aspects. The above described aspects embodiments of the invention are merely descriptive of its principles and are not to be considered limiting. Further modifications of the invention herein disclosed will occur to those skilled in the respective arts and all such modifications are deemed to be within the scope of the invention.

What is claimed is:

1. A collagen cross-linking system for treating a tissue of a patient, the system comprising:
a catheter comprising a flexible shaft and a conforming member, the flexible shaft having a distal end, a proximal end, a shaft body extending between the distal end and the proximal end, the shaft body defining a lumen, the conforming member being fixed to the catheter shaft near the distal end, the conforming member comprising a membrane defining a cavity in fluid communication with the lumen;
a fluid source operatively coupled to the proximal end of the catheter, the flexible shaft of the catheter being adapted and configured so that a flow of photosensitizing fluid provided by the fluid source flows through the membrane of the conforming member; and
a light source operatively coupled to the proximal end of the catheter, the catheter being adapted and configured so that photo-activating light generated by the light source passes through the membrane of the conforming member.

2. The system of claim 1, wherein the conforming member is adapted and configured to apply forces to the tissue, the forces causing the tissue to assume a desired shape.

3. The system of claim 1, wherein the conforming member comprises an inflatable member that expands upon inflation.

4. The system of claim 1, wherein the fluid source comprises a reservoir of photosensitizing fluid.

5. The system of claim 1, wherein the photosensitizing fluid comprises riboflavin.

6. The system of claim 1, wherein the photosensitizing fluid comprises oxygen.

7. The system of claim 1, wherein the light source generates ultraviolet light.

8. The system of claim 1, wherein the light source generates light having a wavelength of 370 nm.

9. A method for the medical treatment for treating a target tissue in a patient's body, the method comprising:
positioning a conforming member near the target tissue, the conforming member comprising a membrane;
directing a flow of photosensitizing fluid to impinge upon the target tissue, wherein the photosensitizing fluid flows through the membrane of the conforming member; and
irradiating the target tissue with photo-activating light that passes through the membrane of the conforming member.

10. The method of claim 9, further comprising inflating the conforming member.

11. The method of claim 9, further comprising stretching the tissue so that the tissue assumes a stretched shape.

12. The method of claim 9, further comprising compressing the tissue so that the tissue assumes a compressed shape.

13. The method of claim 9, further comprising shielding portions of the target tissue so that the target tissue comprises a pattern of unshielded portions and shielded portions.

14. The method of claim 13, wherein the pattern of unshielded portions comprises a plurality of unshielded lines arranged in a pattern analogous to the lines seen on a pumpkin.

15. The method of claim 13, wherein the pattern of unshielded portions comprises a plurality of unshielded lines arranged in a crisscross pattern.

16. The method of claim 9, wherein irradiating the target tissue with photo-activating light comprises irradiating the target tissue with pattern of photo-activating light.

17. The method of claim 9, wherein the photo-activating light comprises ultraviolet light.

18. The method of claim 9, wherein the photo-activating light comprises light having a wavelength of 370 nm.

19. The method of claim 9, wherein the photosensitizing fluid comprises riboflavin.

20. The method of claim 9, wherein the photosensitizing fluid comprises oxygen.
